SEQUENCE LISTING (19 sequences)

SEQ ID NO: 1 - protein, 8 aa, Mus musculus
GYTFTDYY

SEQ ID NO: 2 - protein, 8 aa, Mus musculus
IYPGSGNT

SEQ ID NO: 3 - protein, 9 aa, Mus musculus
VRDSPFFDY

SEQ ID NO: 4 - protein, 12 aa, Mus musculus
QSLLNSGMRKSF

SEQ ID NO: 5 - protein, 3 aa, Mus musculus
WAS

SEQ ID NO: 6 - protein, 8 aa, Mus musculus
KQSYHLFT

SEQ ID NO: 7 - protein, 116 aa, artificial sequence
  humanized heavy chain amino acid sequence of neutralizing anti-PlGF antibody
QVQLQQSGAELVKPGASVKISCKASGYTFTDYYINWVKLAPGQGLEWIGWIYPGSGNTKYNEKFKGKATLTIDTSSSTAYMQLSSLTSEDTAVYFCVRDSPFFDYWGQGTLLTVSS

SEQ ID NO: 8 - protein, 112 aa, artificial sequence
  humanized light chain amino acid sequence of neutralizing anti-PlGF antibody
DIVLTQSPDSLAVSLGERVTMNCKSSQSLLNSGMRKSFLAWYQQKPGQSPKLLIYWASTRESGVPDRFTGSGSGTDFTLTISSVQAEDVAVYYCKQSYHLFTFGSGTKLEIK

SEQ ID NO: 9 - protein, 256 aa, artificial sequence
  humanized scFv amino acid sequence of neutralizing anti-PlGF antibody
QVQLQQSGAELVKPGASVKISCKASGYTFTDYYINWVKLAPGQGLEWIGWIYPGSGNTKYNEKFKGKATLTIDTSSSTAYMQLSSLTSEDTAVYFCVRDSPFFDYWGQGTLLTVSSGGGGSGGGGSGGGGSDIVLTQSPDSLAVSLGERVTMNCKSSQSLLNSGMRKSFLAWYQQKPGQSPKLLIYWASTRESGVPDRFTGSGSGTDFTLTISSVQAEDVAVYYCKQSYHLFTFGSGTKLEIKGSYPYDVPDYAGS

SEQ ID NO: 10 - protein, 152 aa, Homo sapiens
LPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 11 - protein, 21 aa, Homo sapiens
RRPKGRGKRRREKQRPTDCHL

SEQ ID NO: 12 - protein, 121 aa, Mus musculus
  MISC_FEATURE: VH 5D11D4
QVQLQQPGAELVRPGASVKLSCKASGYTFTNYWINWVKQRPGQGLEWIGNIYPSDSFTNYNQKFKDKATLTVDKSSSTAYMHLSSPTSDDSAVYYCTRDYRYDAVYALDYWGQGTSVTVSS

SEQ ID NO: 13 - protein, 106 aa, Mus musculus
  MISC_FEATURE: VL 5D11D4
QIVLTQSPAIMSASPGEKVTITCSASSSVSFIHWFQQKPGTSPKLWIYGTSNLASGVPARFSGSGSGTSSSLTISRMEAEDAATYYCQQRSRYPYTFGGGTKLEIK

SEQ ID NO: 14 - protein, 5 aa, Mus musculus
  MISC_FEATURE: VH CDR1 5D11D4
NYWIN

SEQ ID NO: 15 - protein, 17 aa, Mus musculus
  MISC_FEATURE: VH CDR2 5D11D4
NIYPSDSFTNYNQKFKD

SEQ ID NO: 16 - protein, 12 aa, Mus musculus
  MISC_FEATURE: VH CDR3 5D11D4
DYRYDAVYALDY

SEQ ID NO: 17 - protein, 10 aa, Mus musculus
  MISC_FEATURE: VL CDR1 5D11D4
SASSSVSFIH

SEQ ID NO: 18 - protein, 7 aa, Mus musculus
  MISC_FEATURE: VL CDR2 5D11D4
GTSNLAS

SEQ ID NO: 19 - protein, 9 aa, Mus musculus
  MISC_FEATURE: VL CDR3 5D11D4
QQRSRYPYT

(19) United States
(12) Patent Application Publication
(10) Pub. No.: US 2019/0031762 A1
(43) Pub. Date: Jan. 31, 2019

(54) POSTERIOR OCULAR FIBROSIS INHIBITION BY ANTAGONIZING PLACENTAL GROWTH FACTOR

(71) Applicant: THROMBOGENICS NV, Leuven (BE)

(72) Inventors: Tine Van Bergen, Leuven (Heverlee) (BE); Bart Jonckx, Leuven (Heverlee) (BE); Jean Feyen, Leuven (Heverlee) (BE)

(73) Assignee: THROMBOGENICS NV, Leuven (BE)

(21) Appl. No.: 16/077,013

(22) PCT Filed: Mar. 10, 2017
(86) PCT No.: PCT/EP2017/055652

§ 371 Date: Aug. 9, 2018

(30) Foreign Application Priority Data
Mar. 10, 2016 (EP) 16159687.9

(51) Int. Cl. C07K 16/28; A61P 27/02; C07K 16/22

(52) U.S. Cl. CPC C07K 16/2863; A61P 27/02; C07K 16/22; A61K 45/06; C07K 2317/76; A61K 2300/00; C07K 2317/24

(57) ABSTRACT

The disclosure is situated in the field of ocular therapies. In particular, in some embodiments the disclosure provides antagonists of placental growth factor and related methods of use for treating, preventing, or delaying posterior ocular fibrosis. In other embodiments, the disclosure provides monospecific placental growth factor (PlGF) antagonist compositions and related methods for maintaining or improving visual acuity of a subject with an eye of which the retina is damaged.

### FIELD OF THE INVENTION

The invention is situated in the field of ocular therapies. In particular it refers to antagonists of placental growth factor for interfering with posterior ocular fibrosis.

### BACKGROUND TO THE INVENTION

The retina of an eye (most posterior segment of the eye; back of the eye) is part of the central nervous system (CNS). As such the retina's wound-healing response is similar to the wound-healing response of the brain which Friedlander refers to as gliosis (fibrosis mediated by glial cells). This in contrast to wound-healing responses in non-CNS tissues or organs in general and anterior ocular segments (front of the eye) such as cornea and trabecular meshwork specifically, which is referred to as fibrosis (fibrosis mediated by fibroblasts) (Friedlander 2007, J Clin Invest, 117:576-586).

Any type of retinal disease or disorder accompanied by or caused by inflammation and/or neovascularization leads to gliosis and fibrous scarring. Ultimately, this gliosis, or posterior ocular fibrosis, leads to severe vision loss and blindness. Although a number of drugs are available to suppress neovascularization (e.g. pegaptanib sodium and ranibizumab; and, off-label, bevacizumab; all targeting vascular endothelial growth factor, VEGF), these do not minimize gliosis/posterior ocular fibrosis (Friedlander, J Clin Invest 2007, 117:576-586). It is described that long-term use of anti-VEGF therapy can even lead to increased posterior ocular fibrosis. From the CATT-trial, for instance, it is known that 24.7% of the patients with age-related macular edema (AMD) will develop a posterior fibrotic scar after 2 years of anti-VEGF therapy (bevacizumab or ranibizumab) (Daniel et al., Ophthalmology 2014, 121:656-666).

Recently, Van Bergen et al. (Invest Ophthalmol Vis Sci 2015, 56:5280-5289) used the experimental murine model of laser-induced choroidal neovascularization (CNV) to demonstrate reduction of posterior ocular fibrosis by means of antibodies targeting LOX (lysyl oxidase) or LOXL2 (lysyl oxidase-like 2). In a similar model, Rakic et al. (Invest Ophthalmol Vis Sci 2003, 44:3186-3193) identified placental growth factor (PlGF) as one of the growth factors contributing to CNV, more in particular contributing to neovascularization and lesion size 14 days after inducing laser injury. Hollborn et al. (Graefe's Arch Clin Exp Ophthalmol 2006, 244:732-741) determined that in vitro grown human retinal pigment epithelial (RPE) cells stimulated by transforming growth factor-β (TGF-β) produce increased amounts of PlGF and VEGF, leading to the suggestion that during diabetic retinopathy, TGF-β-caused PlGF-secretion by RPE cells may contribute to cell migration as part of the formation of fibrovascular membranes. The presence of myofibroblasts in these membranes can cause tractional retinal detachment and retinal hemorrhage.

Cao et al. (Invest Ophthalmol Vis Sci 2010, 51:6009-6017) investigated the effect of a VEGF-Trap (binding both VEGF and PlGF) on CNV induced by subretinal injection of Matrigel. The authors observed arrested CNV growth and reduced inflammatory and fibrotic responses. Matrigel, however, contains several growth factors including basic fibroblast growth factor (bFGF), epidermal growth factor (EGF), insulin-like growth factor 1 (IGF-1), TGF-β, platelet-derived growth factor (PDGF), nerve growth factor (NGF), and connective tissue growth factor (CTGF) (Hughes et al., Proteomics 2010, 10:1886-1890). Results obtained with this model can therefore not be compared to results obtained with the laser-induced CNV model which does not introduce an external cocktail of growth factors in the eye.

A beneficial effect of PlGF-neutralizing antibodies has been described for many disorders including pathological angiogenesis, pathological arteriogenesis, inflammation, tumor formation, vascular leakage, and pulmonary hypertension (WO 01/85796), osteoporosis (WO 2004/002524), tissue adhesion (WO 03/063904), liver cirrhosis (WO2007/003609), Philadelphia chromosome positive leukemia (WO 2010/037864) and trabeculectomy outcome (WO 2013/07971); see also Fischer et al. (Cell 2007, 131:463-475), Van Steenkiste et al. (Gastroenterology 2009, 137:2112-2124), Coenegrachts et al. (Cancer Research 2010, 70:6537-6547), Van de Veire et al. (Cell 2010, 141:178-190), Schmidt et al. (Cancer Cell 2011, 19:740-53), Snuderl et al. (Cell 2013, 152:1065-1076), Van Bergen et al. (J Cell Mol Med 2013, 17:1632-1643).

Specifically, Van de Veire et al. (2010) noted inhibition by PlGF-neutralizing antibodies of ocular angiogenesis, ocular inflammation and choroidal vessel leakage after laser-induced CNV (thus in part confirming and extending the data of Rakic et al., Invest Ophthalmol Vis Sci 2003, 44:3186-3193).

The beneficial effect of PlGF-neutralizing antibodies on post-operative tissue adhesion (WO 03/063904) and failure of trabeculectomy Van Bergen et al. (J Cell Mol Med 2013, 17:1632-1643) may, at least in part, be attributed to apparent inhibition of fibroblast-mediated fibrosis.

Scarring (fibrosis) is thought to contribute to bleb failure after glaucoma filtration surgery/trabeculectomy (Li et al. 2006; Free papers Glaucoma: microbiology and bloodflow and IOP; Role of vascular endothelial growth factor and placental growth factor in glaucoma and scar formation after glaucoma filtration surgery). In this context, it was shown that an antibody blocking the VEGF-R2 receptor did, albeit to a lower extent than a PlGF-neutralizing antibody, increase bleb survival (decrease scarring) after glaucoma filtration surgery/trabeculectomy (WO 2013/07971). Both an antibody blocking the VEGF-R2 receptor and a PlGF-neutralizing antibody were thus capable of reducing anterior ocular fibrosis. Friedlander (J Clin Invest 2007, 117:576-586) summarized the lack of action of VEGF-antagonists on posterior ocular fibrosis. The difference in action of VEGF-antagonists on posterior and anterior ocular fibrosis indicates a difference in the processes between posterior ocular fibrosis and anterior ocular fibrosis.

### SUMMARY OF THE INVENTION

The invention relates to monospecific placental growth factor (PlGF) antagonists for use in treating, preventing, or delaying progression of ocular posterior fibrosis in a subject. Alternatively, the monospecific placental growth factor (PlGF) antagonist is for use in treating, preventing, or delaying progression of ocular posterior fibrosis without inducing ocular posterior neurodegeneration in a subject. As a further embodiment the monospecific placental growth factor (PlGF) antagonist for the above uses envisages further treating, preventing, or delaying progression of ocular posterior inflammation and/or ocular posterior neovascularization and/or vessel leakage and/or for use in maintaining or improving the visual acuity of a subject with an eye of which the retina is damaged.

The invention further relates to monospecific placental growth factor (PlGF) antagonists for use in maintaining or improving the visual acuity of a subject with an eye of which the retina is damaged.

In any of the above, the monospecific placental growth factor (PlGF) antagonist alone can be administered to an eye.

Alternatively, in the above uses, the monospecific placental growth factor (PlGF) antagonist may be administered to an eye after wash out of a vascular endothelial growth factor (VEGF) antagonist or a VEGF-receptor (VEGFR) antagonist previously administered to the same eye.

In a further alternative, a vascular endothelial growth factor (VEGF) antagonist or a VEGF-receptor (VEGFR) antagonist is administered to an eye after wash out of the monospecific placental growth factor (PlGF) antagonist previously administered to the same eye.

Further alternatives are envisaged including the combined administration of a monospecific placental growth factor (PlGF) antagonist and a second active compound. As such, in any of the above-described uses, the monospecific placental growth factor (PlGF) antagonist may be administered to an eye in combination with a second active compound wherein said second active compound is different from a vascular endothelial growth factor (VEGF) antagonist and different from a VEGF-receptor (VEGFR) antagonist.

Alternatively, the monospecific placental growth factor (PlGF) antagonist is administered to an eye in combination with a second active compound wherein said second active compound is different from a vascular endothelial growth factor (VEGF) antagonist and different from a VEGF-receptor (VEGFR) antagonist; and wherein said administration is after wash out of a vascular endothelial growth factor (VEGF) antagonist or VEGF-receptor (VEGFR) antagonist previously administered to the same eye.

In a further alternative, a vascular endothelial growth factor (VEGF) antagonist or a VEGF-receptor (VEGFR) antagonist is administered to an eye after wash out of the monospecific placental growth factor (PlGF) antagonist previously administered to the same eye in combination with a second active compound wherein said second active compound is different from a vascular endothelial growth factor (VEGF) antagonist and different from a VEGF-receptor (VEGFR) antagonist.

When a monospecific placental growth factor (PlGF) antagonist is combined with a second active compound, both can be administered to the eye each in a separate composition. The second active agent can be administered prior to, concurrent with, or after the administration of the monospecific placental growth factor (PlGF) antagonist.

Alternatively, both can be administered to the eye combined in a single composition. Second active compounds in this context may be one active compound or a combination of more than one active compound. In particular, but not limiting, such second active compound may be an anti-inflammatory compound, an anti-angiogenic compound, an anti-fibrotic compound, an AGE-inhibiting compound, an ALE-inhibiting compound, an AGE-breaking compound, a carbonic anhydrase inhibitor, an NMDA-receptor antagonist, a kainate receptor antagonist, an AMPA-receptor antagonist, a neuroprotective agent, an agent for controlling the intra-ocular pressure, an anti-apoptotic agent, an antiviral compound, an antibiotic compound, an antifungal compound, an antihistamine, an anticoagulant, a thrombolytic compound, an anti-mitotic agent, an anesthetic agent, and agent inducing mydriasis, an agent inducing cycloplegia, an agent inducing posterior vitreous detachment (complete or incomplete), an agent inducing vitreous liquefaction, an integrin inhibitor, an anti-edema agent.

Any use of the monospecific placental growth factor (PlGF) antagonist as hereinabove described may further be combined with photodynamic therapy, laser photocoagulation, radiation therapy or vitreal surgery.

The monospecific placental growth factor (PlGF) antagonist for any use as described hereinabove may be further characterized in that the posterior ocular fibrosis is occurring concurrent with or after retinal damage. Such posterior ocular fibrosis may for instance be occurring in age-related macular edema, diabetic retinopathy, (diabetic) macular edema, any type of retinopathy, neovascular glaucoma, retinal detachment or retinal hemorrhage.

The monospecific placental growth factor (PlGF) antagonist for any use as described hereinabove may be further characterized in that it is a PlGF-neutralizing antibody or a PlGF-neutralizing fragment of an antibody, an antisense RNA, a small interfering RNA, an aptamer, or a ribozyme. Herein, a PlGF-neutralizing antibody or a PlGF-neutralizing antibody fragment may be one comprising the 6 CDRs comprised in the heavy chain defined in SEQ ID NO:7 and in the light chain defined in SEQ ID NO:8. In particular, these CDRs are as defined in SEQ ID NOs: 1 to 6 when applying the IMGT-method to SEQ ID NOs:7 and 8.

The invention also relates to an isolated PlGF-neutralizing antibody, or a PlGF-neutralizing antibody fragment thereof, comprising the 3 heavy chain CDRs comprised in the heavy chain defined in SEQ ID NO:12 and the 3 light chain CDRs comprised in the light chain defined in SEQ ID NO:13.

### FIGURE LEGENDS

FIG. 1. Leukocyte infiltration in the laser-induced CNV model.

(A) In the CNV model, 5 days after lasering, treatment with anti-PlGF antibody 5D11D4 (1.5 and 3.1 μg) decreased leukocyte infiltration as compared to IgG-treated mice (P<0.05); aflibercept (2.4 and 20 μg) and the high-dose of triamcinolone acetonide (TAAC; 40 μg) showed comparable effects. Anti-VEGFR2 antibody DC101 administration (3.1 μg) did not show any anti-inflammatory effects. (B) Equimolar comparison 5 days after lasering. Treatment with anti-PlGF antibody 5D11D4 (3.1 μg) decreased leukocyte infiltration as compared to IgG treated mice (P<0.05); equimolar concentrations of aflibercept (Eylea®) (2.4 μg) showed similar effect. Anti-VEGFR2 antibody DC101 (3.1 μg) and TAAC administration (4 μg) did not show any anti-inflammatory effects. Data are mean±SEM.

FIG. 2. Posterior ocular collagen deposition in the laser-induced CNV model.

(A) In the CNV model, 30 days after lasering, as compared to PBS treated mice, treatment with anti-PlGF antibody 5D11D4 (1.5 and 3.1 μg) decreased collagen deposition (P<0.05); which was similar to the effect of a high-molar concentration of triamcinolone acetonide (TAAC; 40 μg). Anti-VEGFR2 antibody DC101 and aflibercept (Eylea®) administration (both 3.1 μg) did not show any anti-fibrotic effects. (B) Equimolar comparison 30 days after lasering. Treatment with anti-PlGF antibody 5D11D4 (3.1 μg) decreased collagen deposition as compared to IgG treated mice (P<0.05). Anti-VEGFR2 antibody DC101 (3.1 μg), aflibercept (2.4 μg) and TAAC administration (4 μg) did not show any anti-fibrotic effects. Data are mean±SEM.

FIG. 3. Retinal ganglion cell (RGC) survival.

Retinal ganglion cell survival was assessed after 2 (FIG. 3A), 4 (FIG. 3B) and 6 weeks (FIG. 3C) of intraperitoneal injections with control IgG, anti-PlGF antibody 5D11D4 and anti-VEGF-R2 antibody DC101 (all 25 mg/kg, 3 times per week). The RGCs/retinal area was not significantly different between the 3 treatment groups after 2, 4 and 6 weeks in C57Bl/6 mice (N=6; P>0.05), whereas a significant reduction was present in Swiss mice (N=6; P<0.05). As shown in FIG. 3D, TUNEL staining confirming the number of apoptotic cells per retinal area in the ganglion cell layer was comparable in the anti-PlGF antibody 5D11D4 versus control IgG treated mice after 6 weeks (N=6; P>0.05). A trend of increase in apoptotic cells was present for the anti-VEGF-R2 antibody DC101 treated C57Bl/6 mice (n=6; P=0.10), whereas the increase was significant in the Swiss group (n=6; P<0.001). Data represent mean±SEM.

FIG. 4. Posterior ocular collagen deposition in the laser-induced CNV mouse model. Posterior ocular collagen deposition, compared PBS treated eyes, was assessed after intravitreal administration of anti-mouse PlGF antibody 5D11D4, of anti-human PlGF antibody 16D3, of anti-murine VEGF antibody B20 (all 3.1 μg/eye), of aflibercept (equimolar amount of 2.4 μg/eye), and of triamcinolone acetonide (TAAC; 40 μg/eye). Both anti-PlGF antibodies significantly decreased collagen deposition (P<0.05); which was similar to administration of TAAC (40 μg/eye). Administration of an equimolar amount of aflibercept or of anti-VEGF antibody B20 did not reduce fibrosis compared to PBS treated eyes (P<0.05). Data represent mean±SEM.

FIG. 5. RGC density in eyes of diabetic mice (streptozotocin-induced diabetes).

Eight weeks after the onset of diabetes, the number of RGCs (250 μm from either side of optic nerve) was not significantly different between eyes receiving no treatment (---), receiving intravitreal administration of anti-PlGF antibody 5D11D4 (5.4 μg/eye), or receiving PBS injection. In contrast, administration of anti-VEGFR2 antibody DC101 significantly reduced the RGC density with 20% (P<0.05). Data represent mean±SEM.

FIG. 6. Pericyte coverage in retinal vessels in the laser-induced CNV mouse model. Treatment with anti-PlGF antibody 5D11D4 (25 mg/kg) increases vessel maturation in CNV as analysed at day 14 after lasering. Intraperitoneal administration of anti-PlGF antibody (3 times per week) started immediately after lasering and until upon sacrifice. Treatment with anti-PlGF antibody 5D11D4 (25 mg/kg) increased the αSMA (smooth muscle cell actin) positive area, as compared to treatment with control IgG antibody 1C8 (“IgG”, n=10, P<0.05). The effect of administration of anti-VEGF-R2 antibody DC101 (25 mg/kg) treatment was not significant, as compared to administration of control IgG (n=10, P>0.05). Data represent mean±SEM.

### DETAILED DESCRIPTION OF THE INVENTION

In work leading to the invention, the effect of different angiogenesis-inhibitors on different aspects of laser-induced choroidal neovascularization (CNV) was compared.

The angiogenesis-inhibitors involved are an antibody blocking the vascular endothelial growth factor receptor 2 (VEGF-R2) (receptor of VEGF-A), a murine placental growth factor (PlGF)-neutralizing antibody (as described in WO 01/85796; and see below), a human PlGF-neutralizing antibody (as described in WO 2006/099698; and see below), an anti-murine VEGF antibody B20 (Liang et al. 2006, J Biol Chem 281:951-961), and aflibercept (capturing both VEGF-A, VEGF-B and PlGF; tradename Eylea®). The aspects of CNV that were studied are inflammation, neovascularization, vessel leakage (including effect on vessel pericytes), and posterior ocular fibrosis. The effect on retinal ganglion cells was investigated in naive mice and in a diabetic mouse model. The murine antibody against VEGFR-2 and against PlGF, as well as aflibercept all reduced neovascularization and vessel leakage. Strikingly, only aflibercept and the PlGF-neutralizing antibody were able to reduce inflammation (comparable reduction at comparable dose), whereas the VEGF-R2 receptor-blocking antibody did not reduce inflammation. The inflammation-reducing effect of aflibercept thus is attributable to its PlGF-capturing feature. In relation to PlGF-neutralizing antibodies, these data confirm earlier published observations (Van de Veire et al., Cell 2010, 141:178-190).

In striking contrast therewith, however, the current work identified PlGF inhibitors as only agents being able to reduce posterior ocular fibrosis. Such effect was not seen with aflibercept, neither with an antibody blocking VEGF, nor with an antibody blocking the VEGF-R2 receptor. This is very surprising as aflibercept, although able to neutralize PlGF, did not reduce posterior ocular fibrosis. This goes against the sometimes conceived or expressed conviction that PlGF and VEGF are just alternative growth factor acting similarly in the VEGF-VEGFR pathways. It is furthermore surprising as these results, in confirming the difference between posterior ocular fibrosis and anterior ocular fibrosis, indicate that blocking PlGF action in the back of the eye holds potential for halting posterior ocular fibrosis, this in contrast to VEGF-inhibition (potentially increasing posterior ocular fibrosis, see Background section).

Observed further in this work was a lack of toxicity of a PlGF antagonist on RGCs, this in contrast to a significant RGC apoptosis rate induced by an antibody blocking the VEGF-R2 receptor.

In view of the above, the invention therefore relates to monospecific placental growth factor (PlGF) antagonists for use in treating, preventing, or delaying progression of ocular posterior fibrosis in a subject. Alternatively, the monospecific placental growth factor (PlGF) antagonist is for use in treating, preventing, or delaying progression of ocular posterior fibrosis without inducing ocular posterior neurodegeneration in a subject. As a further embodiment the monospecific placental growth factor (PlGF) antagonist for the above uses envisages further treating, preventing, or delaying progression of ocular posterior inflammation and/or ocular posterior neovascularization and/or vessel leakage.

Ocular posterior fibrosis is associated with the healing of any retinal wound, damage, or trauma (collectively referred to herein as retinal damage). Fibrosis occurring due to the healing response/process occurring at the back of the eye (posterior zone of the eye) is referred to as gliosis (fibrosis mediated by glial cells) by Friedlander (J Clin Invest 2007, 117:576-586), see also the Background section hereinabove.

A specific antagonist is an antagonist that blocks, neutralizes or otherwise abolishes (e.g. inhibits) the action of the antagonist's target molecule, and not, or not significantly, the action of another molecule (therewith a non-target molecule). The blocking, neutralization or otherwise abolishing of the action of the target molecule thus is selective. The blocking, neutralization or otherwise abolishing of the action of the target molecule can be partial (e.g. anywhere between 5% and 95% residual activity left=anywhere between 95% and 5% inhibition) or near complete (e.g. more than 95% inhibition).

In case of a ligand-receptor interaction, the ligand can be the sole ligand of a (not necessarily sole) receptor; or multiple ligands can bind to the same receptor in which case all or some ligands may bind to the same site of the receptor, or all or some ligands each may bind to a different site of the receptor. Specific antagonism of a ligand is always possible. In case of specific receptor inhibition, this would be possible by targeting either in case of a sole receptor or in case of targeting a unique binding site in the receptor for a target ligand.

The blocking, neutralization or otherwise abolishing of the action of the target molecule by a selective antagonist usually implies physical interaction between the antagonist and the target molecule. This does not exclude binding of the selective antagonist to non-target molecules but the (biological) action of latter should then not be, or not significantly be, blocked, neutralized or otherwise abolished. Alternatively, the (biological) action of the target molecule is inhibited to a much higher extent, e.g. 25-fold, 50-fold, 100-fold or more, compared to the inhibition of the non-target molecule, thus creating selectivity. Comparison of inhibition can be expressed e.g. in terms of concentration of the antagonist required to inhibit 50% of the (biological) activity of a molecule (IC50 value).

In particular, a specific antagonist is a monospecific antagonist. This implies that the antagonist is targeting (in the sense of blocking, neutralizing, or otherwise abolishing the action as described above) only one specific molecule. This does not exclude multivalency of the (mono)specific antagonist. Such antagonist thus could have multiple binding sites, each of these interacting with the same part of the molecule; or each of these, or some of these interacting with distinct parts of the target molecule. In toto, however, the antagonist is specific, or monospecific, for one and the same molecule, i.e. the same target molecule. The concept of specificity and monospecificity furthermore extends to multiple isoforms of a molecule. For instance, bevacizumab is a monoclonal antibody inhibiting multiple isoforms of vascular endothelial growth factor A (VEGF-A) and is therefore a monospecific VEGF-A antagonist.

Although the concept of monospecificity is well-known in the antibody field, it extends to small molecules (e.g. class I retinoids are monospecific (ant)agonists of only one type of retinoic acid receptor, this compared to class II retinoids that are non-specific—Géhin et al., Chem Biol 1999, 6:519-529), as well as to e.g. antisense oligonucleotides, siRNAs, and aptamers (traditionally monospecific, but bispecific antisense oligonucleotides, siRNAs, and aptamers are known, e.g., Rubenstein & Guinan, In vivo 2010, 24:489-494; Anderson et al., Oligonucleotides 2003, 13:303-312; and Schrand et al., Cancer Immunol Res 2014, 2:867-877, respectively). A trivalent but otherwise monospecific ribozyme has been described by Bai et al. (AIDS Res Hum Retrovir 2001, 17:385-399).

Human placental growth factor, hPlGF, was first disclosed by Maglione et al. (Proc Natl Acad Sci USA 1991, 88:9267-9271) and refers to 4 isoformic variants of the polypeptide accessible under GenBank accession no. P49763, of which PlGF1 and PlGF2 (also referred to as PlGF-1 and PlGF-2) are the most well-known. The full-length reference sequence of human PlGF-2 (i.e. the mature protein lacking the 18-amino acid signal sequence; hPlGF2) is included hereafter:

(SEQ ID NO: 10)
LPAVPPQQWALSAGNGSSEVEVVPFQEVWGRSYCRALERLVDVVSEYPSE
VEHMFSPSCVSLLRCTGCCGDENLHCVPVETANVTMQLLKIRSGDRPSYV
ELTFSQHVRCECRPLREKMKPERRRPKGRGKRRREKQRPTDCHLCGDAVP
RR.

Compared to hPlGF2, the heparin binding domain with sequence RRPKGRGKRRREKQRPTDCHL (SEQ ID NO: 11) is absent in hPlGF1. The alternative abbreviation “PGF” for placental growth factor is often being used nowadays. In the context of monospecific antagonists of PlGF, such monospecificity thus can extend to all isoforms of PlGF. A “specific inhibitor of PlGF” as used herein thus is a molecule or compound that inhibits the function of PlGF, inhibits PlGF expression or inhibits PlGF signaling without interfering with, or without significantly interfering with (selectively interfering with), the physiological function of other molecules. In particular, a selective PlGF inhibitor will not interfere with the function of VEGF. Thus, as a non-limiting example, a compound specifically directed against PlGF (e.g. an anti-PlGF antibody) is a (mono)specific inhibitor, while compounds that also target VEGF (such as VEGFR1-based compounds and VEGF-Trap or VEGF-Trap-like compounds) or target VEGF/PlGF-shared receptors (e.g. an antibody against VEGFR1, or sVEGFR-1) is typically a non-specific inhibitor as these are not (mono)specific PlGF antagonists. VEGF antagonists and VEGF-receptor antagonists thus are not (mono)specific PlGF antagonists.

PlGF-neutralizing antibodies have been disclosed in for instance WO 01/85796, WO 2006/099698 (see also Nielsen & Sengelov, Expert Opin Biol Ther 2012, 12:795-804), WO 2011/088111 and by e.g. Bais et al. (Cell 2010, 141:166-177—one of these, C9.V2 being used by Snuderl et al., Cell 2013, 152:1065-1076). In particular, the human PlGF-neutralizing antibody 16D3 disclosed in WO 2006/099698 comprises VH CDR1 with sequence GYTFTDYY (SEQ ID NO:1), VH CDR2 with sequence IYPGSGNT (SEQ ID NO:2), VH CDR3 with sequence VRDSPFFDY (SEQ ID NO:3), VL CDR1 with sequence QSLLNSGMRKSF (SEQ ID NO:4), VL CDR2 with sequence WAS (SEQ ID NO:5), and VL CDR3 with sequence KQSYHLFT (SEQ ID NO:6). The hybridoma expressing the murine antibody was deposited by Thromb-X (Herestraat 49, B-3000 Leuven) with the BCCM/LMBP (Belgian Co-ordinated Collections of Microorganisms/Plasmid Collection Laboratorium voor Moleculaire Biologie), University of Ghent, Technologiepark 927, B-9052 Zwijnaarde, Belgium, on 29 Mar. 2005 with biological deposit accession number LMBP 6399CB.

Humanized VH, VL, and scFv amino acid sequences exemplified in WO 2006/099698 are: Humanized VH amino acid sequence:

(SEQ ID NO: 7)
QVQLQQSGAELVKPGASVKISCKASGYTFTDYYINWVKLAPGQGLEWIGW
IYPGSGNTKYNEKFKGKATLTIDTSSSTAYMQLSSLTSEDTAVYFCVRDS
PFFDYWGQGTLLTVSS

Humanized VL amino acid sequence:

(SEQ ID NO: 8)
DIVLTQSPDSLAVSLGERVTMNCKSSQSLLNSGMRKSFLAWYQQKPGQSP
KLLIYWASTRESGVPDRFTGSGSGTDFTLTISSVQAEDVAVYYCKQSYHL
FTFGSGTKLEIK

Humanized scFv amino acid sequence (6-His tag omitted compared to sequence in WO 2006/099698):

(SEQ ID NO: 9)
QVQLQQSGAELVKPGASVKISCKASGYTFTDYYINWVKLAPGQGLEWIGW
IYPGSGNTKYNEKFKGKATLTIDTSSSTAYMQLSSLTSEDTAVYFCVRDS
PFFDYWGQGTLLTVSSGGGGSGGGGSGGGGSDIVLTQSPDSLAVSLGERV
TMNCKSSQSLLNSGMRKSFLAWYQQKPGQSPKLLIYWASTRESGVPDRFT
GSGSGTDFTLTISSVQAEDVAVYYCKQSYHLFTFGSGTKLEIKGSYPYDV
PDYAGS

The murine PlGF-neutralizing antibody 5D11D4 as used in WO 01/85796 is characterized by the heavy- and light chain amino acid sequences given hereafter.

Heavy chain 5D11D4: FR1-CDR1-FR2-CDR2-FR3-CDR3-FR4

(SEQ ID NO: 12)
QVQLQQPGAELVRPGASVKLSCKASGYTFTNYWINWVKQRPGQGLEWIGN
IYPSDSFTNYNQKFKDKATLTVDKSSSTAYMHLSSPTSDDSAVYYCTRDY
RYDAVYALDYWGQGTSVTVSS,

wherein CDR H1, CDR H2, and CDR H3 are defined by the amino acid sequences NYWIN (SEQ ID NO:14), NIYPSDSFTNYNQKFKD (SEQ ID NO:15), and DYRYDAVYALDY (SEQ ID NO:16), respectively.

Light chain 5D11D4: FR1-CDR1-FR2-CDR2-FR3-CDR3-FR4

(SEQ ID NO: 13)
QIVLTQSPAIMSASPGEKVTITCSASSSVSFIHWFQQKPGTSPKLWIYGT
SNLASGVPARFSGSGSGTSSSLTISRMEAEDAATYYCQQRSRYPYTFGGG
TKLEIK,

wherein CDR L1, CDR L2, and CDR L3 are defined by the amino acid sequences SASSSVSFIH (SEQ ID NO:17), (SEQ ID NO:18), and QQRSRYPYT (SEQ ID NO:19), respectively.

The above murine PlGF-neutralizing antibody, as well as PlGF-neutralizing fragments thereof, as well as humanized versions of such antibody or antibody fragment, form a further aspect of the current invention. In particular, the invention relates to an isolated PlGF-neutralizing antibody, or a PlGF-neutralizing antibody fragment thereof, comprising the 3 heavy chain CDRs comprised in the heavy chain defined in SEQ ID NO:12 and the 3 light chain CDRs comprised in the light chain defined in SEQ ID NO:13, wherein the CDRs are delineated by any of the well-known methodologies as described below. In particular, the CDRs as defined in SEQ ID NOs: 14 to 19 where delineated applying the Kabat-method to SEQ ID NOs:12 and 13. Alternatively, the invention relates to a murine PlGF-neutralizing antibody or a murine PlGF-neutralizing antibody fragment competing with 5D11D4 for binding to murine PlGF, or binding to the same murine PlGF-epitope as bound by 5D11D4.

The determination of the CDR regions in an antibody sequence may depend on the algorithm/methodology applied (Kabat-, Chothia-, Martin (enhanced Chothia), IMGT (ImMunoGeneTics information system)-numbering schemes; see, e.g. http://www.bioinf.org.uk/abs/index.html#kabatnum and http://www.imgt.org/IMGTScientificChart/Numbering/IMGTnumbering.html), which can give rise to differences in CDR sequence length and/or -delineation. The CDRs of the anti-PlGF antibodies described in WO 01/85796 and WO 2006/099698 can therefore be alternatively described as the CDR sequences as present in the given respective heavy- and light-chain sequences, and as determined or delineated according to a well-known methodology such as according to the Kabat-, Chothia-, Martin (enhanced Chothia), or IMGT-numbering scheme. The CDR sequences defined in SEQ ID NOs:1 to 6, for instance, have, according to described WO 2006/099698, been delineated from the 16D3 anti-PlGF antibody by means of the IMGT-method. Applying another method may result in CDR sequences (slightly) different from those defined in SEQ ID NOs:1-6.

Herein, a PlGF-neutralizing antibody or a PlGF-neutralizing antibody fragment may be one comprising 6 CDRs of anti-human PlGF antibody 16D3, namely the 3 VH CDRs comprised in the heavy chain defined in SEQ ID NO:7 and the 3VL CDRs comprised in the light chain defined in SEQ ID NO:8, wherein the CDRs are delineated by any of the well-known methodologies as described above. In particular, these CDRs are as defined in SEQ ID NOs: 1 to 6 when applying the IMGT-method to SEQ ID NOs:7 and 8. Outside and flanking the complementarity determining regions, a PlGF-neutralizing antibody or a PlGF-neutralizing antibody fragment may be comprising suitable framework regions (FR), such as those derivable from the VH defined in SEQ ID NO:7 and from the VL defined in SEQ ID NO:8, or any humanized version thereof. Alternatively, the PlGF-neutralizing antibody or a PlGF-neutralizing antibody fragment may be one competing with 16D3 for binding to PlGF, or binding to the same PlGF-epitope as bound by 16D3. The antibody 16D3 binds to human PlGF as well as, albeit with lower affinity, to murine PlGF.

In particular said neutralizing anti-PlGF antibody may be any type of antibody or any fragment of any thereof that is capable of binding to PlGF and of inhibiting an activity of PlGF. In particular, said anti-PlGF antibody or fragment thereof may be neutralizing an activity of PlGF, thus may be a neutralizing anti-PlGF antibody or neutralizing anti-PlGF antibody fragment. Such antibodies include all types of antibodies known in the art, such as human or humanized antibodies, cameloid antibodies, shark antibodies, nanobodies, (single) domain antibodies, miniaturized antibodies (e.g. small modular immunopharmaceuticals, SMIPs), unibodies, etc., and any fragment of any thereof. Exemplary antibody fragments include Fab, F(ab′)2, scFv, scFV-Fc, minibody, V-NAR, VhH. (Nelson, mAbs 2010, 2:77-83; Holliger & Hudson, Nat Biotechnol 2005, 23:1126-1136).

PlGF antisense RNAs are known in the art (e.g. Yonekura et al., J Biol Chem 1999, 274:35172-35178; Levati et al., Int J Oncol 2011, 38:241-247), as well as PlGF siRNA for RNA interference (e.g. Li et al., Oncogene 2013, 32:2952-2962; Nourinia et al., J Ophthalmic Vis Res 2013, 8:4-8) and anti-PlGF ribozymes (e.g. Chen et al., J Cell Biochem 2008, 105:313-320).

A non-exhaustive list of VEGF- and VEGFR-inhibiting compounds is included hereafter. Monospecific VEGF-inhibiting agents include the antibody bevacizumab (binding all VEGF-A isoforms), or antibody fragment ranibizumab (binding all VEGF-A isoforms), the RNA-aptamer pegaptanib (binding only one VEGF-A isoform) and abicipar (VEGF-A-specific designed ankyrin repeat protein (darpin)). Aflibercept is a multipecific inhibitor capturing both VEGF-A, VEGF-B, and PlGF). VEGFR-2(Flk-1) blocking agents include the antibody DC101 (produced by hybridoma cell line ATCC HB-11534). VEGFR-1(Flt-1) blocking agents include peptides (Taylor & Goldenberg 2007, Mol Cancer Ther 6:524-531; Bae et al. 2005, Clin Cancer Ther 11:2651-2661; Ponticelli et al. 2008, J Biol Chem 283:34250-34259) and antibodies (e.g. as described in WO 2006/055809).

“Treatment/treating” refers to any rate of reduction, delaying or retardation of the progress of a disease or disorder, or a single symptom thereof, compared to the progress or expected progress of the disease or disorder, or a single symptom thereof, when left untreated. More desirable, the treatment results in no/zero progress of a disease or disorder (i.e. “inhibition”) or a single symptom thereof, or even in any rate of regression of the already developed disease or disorder, or in any rate of regression of a single symptom of the already developed disease or disorder.

Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. be patient well-being. In the case of quantitative evaluation, the significant amelioration is typically a more than 10%, more than 20%, more than 25%, more than 30%, more than 40%, more than 50%, more than 60%, more than 70%, more than 75%, more than 80%, more than 90%, more than 95%, or a 100% or more improvement over the situation prior to treatment. The time-frame over which the improvement is evaluated will depend on the type of criteria/disease observed and can be determined by the person skilled in the art.

In some instances, a treatment can be prophylactic, meaning that it results in preventing the onset of a disease or disorder, or of a single symptom thereof. In the current context for instance, the development of ocular posterior fibrosis takes time and can in principle starts to occur concurrent with or after any type of retinal damage. If such retinal damage is recognized early enough, then a monoselective PlGF antagonist could be administered as of these early stages to prevent the onset of significant development of ocular posterior fibrosis. Likewise, it is known that in patients diagnosed with retinal damage in one eye (due to a pathology), the fellow or companion eye, although maybe yet healthy, may become subject to the same retinal damage (due to the pathology) (e.g. Stalmans, Graefes Arch Clin Exp Ophthalmol 2016, doi 10.1007/s00417-016-3294-1). In such cases, prophylactic treatment of the fellow or companion eye with a monoselective PlGF antagonist may be considered in order to prevent posterior ocular fibrosis to occur once the retinal damage is a fact. A monoselective PlGF antagonist could in other words be used to prevent ocular posterior fibrosis. Another circumstance in which a monoselective PlGF antagonist could be used to prevent ocular posterior fibrosis is in combination with (e.g. shortly after) surgical vitrectomy. As retinal damage may occur as a side-effect of surgical vitrectomy, it can be envisaged to prevent posterior ocular fibrotic responses to such damage from occurring.

Any damage to the retina can trigger chronic wound healing responses including posterior ocular fibrosis and scarring. Abnormalities in retinal and choroidal vasculature, all damaging the retina, are at the basis of many sight-threatening diseases including age-related macular degeneration, diabetic retinopathy, retinopathy of prematurity, any type of retinopathy, neovascular glaucoma, and macular edema and complications such as vitreomacular traction or symptomatic vitreomacular adhesion (causing fraction of the vitreous on the retina), retinal and vitreous hemorrhage, retinal detachment, macular holes etc. Retinal damage can also be the result of vitreomacular traction or (symptomatic) vitreomacular adhesion, or be the result of neurodegenerative assaults (see further).

Age-related macular degeneration (AMD) is divided in dry AMD (non-neovascular) and wet AMD (neovascular). Wet AMD is characterized by choroidal neovascularization (CNV). In the developed world, AMD is one of the main causes of severe and irreversible loss of central vision, and ultimately, blindness. CNV is often assessed by fluorescent angiography (evidenced by hyperfluorescent proliferating and/or leaking vessels) or by optical coherence tomography (OCT), but the patient's visual acuity determination is the most relevant clinical parameter. CNV can also develop with pathologic myopia or with the ocular histoplasmosis syndrome. Subretinal fibrosis occurs during AMD (Friedlander, J Clin Invest 2007, 117:576-586).

Several ways of treating AMD, in particular wet AMD, exist:
photodynamic therapy (PDT): uses photosensitive drugs (eg, verteporfin) that can be administered systemically (e.g. intravenous), followed by activation with nonthermal light to achieve selective vaso-occlusion of the arteriolarized neovessels, i.e. selective destruction of CNV
anti-inflammatory agents: steroids, corticosteroids or other immunosuppressants, for instance intravitreal, subtenon or subconjunctival dexamethasone, triamcinolone acetonide (TAAC), or fluocinolone acetonide; often these agents also exert antiangiogenic, antifibrotic and antipermeability (anti-edematous) effects. Sustained-release steroid implants (e.g. Ozurdex®, Iluvien®) offer advantages over e.g. multiple intravitreal injections. Other anti-inflammatory agents can target cytokines such as tumor necrosis factor α (TNFα), e.g. infliximab (Olson et al., Arch Ophthalmol 2007, 125:1221-1224) Inhibition of the complement system is another route for obtaining anti-inflammatory effects. Complement system inhibitors include the complement factor C5 inhibiting aptamer avacincaptad pegol sodium (Zimura®); and an inhibitor of the complement factor C3, POT-4, being a derivative of the cyclic peptide compstatin (Querques et al., Ophthalmic Res 2015, 53:194-199).
anti-VEGF agents: bevacizumab (off-label), ranibizumab, aflibercept, pegaptanib sodium; or other such as the DARPin-based abicipar pegol, the single-chain anti-VEGF antibody brolucizumab, the VEGF-Trap variant conbercept (Barakat & Dugel, Retinal Physician 2015, 12:26-36; Querques et al., Ophthalmic Res 2015, 53:194-199); or such as the VEGF-Trap variant VEGF-Grab (Lee et al., Mol Cancer Ther 2015, 14:470-479). Pazopanib, a multi-tyrosine kinase inhibitors blocking VEGFR1-, VEGFR2-, VEGFR3-, PDGFRα and PDGFRβ-receptors is likewise under evaluation (Querques et al., Ophthalmic Res 2015, 53:194-199).
thermal laser ablation, laser photocoagulation
ionizing radiation/radiation therapy (Finger et al., Ophthalmology 1996, 103:878-889)
anti-angiogenic agents other than anti-VEGF agents: agents for instance inhibiting platelet derived growth factor (PDGF), basic fibroblast growth factor (bFGF), transforming growth factor β (TGFβ); or such as squalamine (Barakat & Dugel, Retinal Physician 2015, 12:26-36). Anti-PDGF-B agents under clinical evaluation include the pegylated aptamer pegpleranib sodium (Fovista®) (Querques et al., Ophthalmic Res 2015, 53:194-199).
anti-fibrotic agents: agents for instance inhibiting connective tissue growth factor (CTGF); 5-fluorouracil (5-FU)
combination therapies: PDT+anti-inflammatory agent; PDT+anti-VEGF agent; PDT+anti-inflammatory agent+anti-VEGF agent (Yip et al., Br J Ophthalmol 2009, 93:754-758; Shah et al., Retina 2009, 29:133-148); anti-VEGF agent+anti-PDGF agent (mentioned in Spaide, Retina 2009, 29:S5-S7)
vitreal surgery (surgical excision of subfoveal CNV via pars plana vitrectomy; surgical translocation of the fovea, for subfoveal CNV; the resulting juxtafoveal or extrafoveal CNV can then be treated with standard laser photocoagulation or PDT).

Diabetic retinopathy (DR) is, likewise to AMD, divided in two stages. The early stage is non-neovascular and is termed non-proliferative diabetic retinopathy (NPDR), itself subdivided in mild, moderate, and severe NPDR. The advanced stage is neovascular and termed proliferative diabetic retinopathy (PDR). Vision loss due to (advanced) DR may occur once the macula is affected (“diabetic maculopathy”). Diabetic macular edema (DME) may occur at any DR stage but is more frequently associated with later-stage DR and is characterized by vascular leakage leading to swelling of the macula. Further classifications of diabetic maculopathy include it being central (affecting fovea) or non-central (not affecting fovea); focal or diffuse (depending on extent of edema); ischemic or non-ischemic; and tractional or non-tractional. An important aspect of multifactorial DR is neurodegeneration. (Stitt et al., Prog Retin Eye Res 2016, 51:156-186). Epiretinal fibrosis occurs during DR (Friedlander, J Clin Invest 2007, 117:576-586).

Several ways of treating DR exist (Stitt et al., Prog Retin Eye Res 2016, 51:156-186; Park & Roh, J Diabet Res 2016, article ID 1753584):
controlling diabetes in general (hyperglycemia, dyslipidemia, hypertension, smoking)
when DME occurs: laser photocoagulation (focal or grid laser treatment, or newer concepts such as subthreshold diode micropulse laser photocoagulation (SDM), retinal rejuvenation therapy (2RT) and selective retina therapy (SRT)), anti-VEGF agents, and corticosteroids (e.g. triamcinolone, dexamethasone, fluocinolone) or non-steroidal anti-inflammatory drugs (NSAIDs), or a combination of any of these. See also the section herein on AMD for more elaborate information on anti-VEGFs and anti-inflammatory agents.
when PDR occurs: pan-retinal laser photocoagulation; vitreal surgery (vitrectomy); anti-VEGF agents or steroids to halt further progression

The aim of any treatment is to stabilize the patient's visual acuity (i.e. to prevent further deterioration of visual acuity) but ideally also to improve the patient's visual acuity (VA), this compared to the patient's visual acuity at the onset of the treatment. Different methods for determining VA are discussed by e.g. Vanden Bosch and Wall (Eye 1997, 11:411-417) and computerized methods of VA testing have been introduced (e.g. Beck et al., Am J Ophthalmol 2003, 135:194-205).

The invention therefore also relates to monospecific placental growth factor (PlGF) antagonists for use in maintaining or improving the visual acuity of a subject with an eye of which the retina is damaged.

Retinal ganglion cells (RGCs) and glial cells are vulnerable to metabolic stress conditions. Degeneration of these cells is occurring in ocular pathologies such as diabetic retinopathy (DR), age-related macular degeneration (AMD), and glaucoma. Factors contributing to cell death/apoptosis include advanced glycation endproducts (AGEs), advanced lipoxidation endproducts (ALEs), free radical species, high intraocular pressure (IOP), hypoxia (Schmidt et al., Curr Neuropharmacol 2008, 6:164-178; Barber et al., Prog Neuropsychopharmacol Biol Psychiatry 2003, 27:283-290).

A number of AGE-inhibiting compounds is known, including aminoguanidine (and derivatives thereof), pyridoxamine, 2,3 diaminophenazine (2,3DAP), thiazolidine derivatives (e.g. OPB-9195), carnosine, tenilsetam, thiamine, benfotiamine, “Lalezari-Rahbar” (LR) compounds, and derivatives of edaravone (reviewed in Nagai et al., Diabetes 2012, 61:549-559; see e.g. Table 1 and FIG. 2 in this reference). Other AGE inhibitors include inhibitors of angiotensin converting enzyme (ACE), e.g. ramipril, benazepril, temocaprilat, AVE8048; angiotensin receptor blockers (ARBs), e.g. losartan, valsartan, olmesartan, R147176; and antihypertensive agents, e.g. hydralazine (reviewed in Nagai et al., Diabetes 2012, 61:549-559; see e.g. Table 1 in this reference).

Further known is a number of AGE-breaking compounds, including N-phenacylthiazolium bromide, and a derivative thereof known as ALT-711 or alagebrium, and pyridinium analogs TRC4186 and TRC4149 (reviewed in Nagai et al., Diabetes 2012, 61:549-559; see e.g. Table 1 and FIG. 3 in this reference).

It is believed that most AGE inhibitors are potentially also ALE inhibitors (Baynes & Thorpe, Free Radic Biol Med 2000, 28:1708-1716). ALE inhibitors further include compounds capable of neutralizing ALE precursors generated from lipid peroxidation, e.g. hydrazine and hydrazine derivatives (e.g. hydralazine, dihydralazine, aminoguanidine, OPB-9195), vitamin B6 and vitamin B6 derivatives (e.g. pyridoxamine, pyridoxal isonicotyl hydrazones). Amino-acid derivatives such as carnosine, histidyl hydrazide, N-acetyl cysteine and S-adenosylmethionine have also been considered as ALE-inhibitors. Further ALE-inhibitors include ACE inhibitors (e.g. captotril, enalapril, fosinopril), ARB inhibitors (e.g. losartan, candesartan), and antioxidants. ALE-inhibitors described above were reviewed by Negre-Salvayre et al. (Br J Pharmacol 2008, 153:6-20).

Compounds aimed at reducing apoptosis (anti-apoptotic agents) include carbonic anhydrase blockers (e.g. dorzolamide (Schmidt et al., Br J Ophthalmol 1998, 82:758-762)). Another carbonic anhydrase blocker, i.e. acetazolamide, was disclosed to decrease cystoid macular edema in patients with retinitis pigmentosa as well as in diabetic macular edema (Giusti et al., Int Ophthalmol 2002, 24:79-88).

The excitatory amino acid glutamate released during metabolic stress contributes to the initiation of RGC death through binding to the N-methyl-D-aspartate receptor (NMDA-receptor), in turn leading to excessive levels of intracellular calcium. Blockers of the NMDA-receptor are known to protect RGCs and include MK-801 (dizocilpine; 5-methyl-10,11-dihydro-5H-dipenzocyclohepta-5,10-iminomaleate) (e.g. Weber et al., Graefes Arch Clin Exp Ophthalmol 1995, 233:360-365), memantine (e.g. Vorwerk et al., Invest Ophthalmol Vis Sci 1996, 37:1618-1624), dextromethorphan (Yoon & Marmor, Arch Ophthalmol 1989, 107:409-411), amino-phosphonovaleric acid (DeVries & Schwartz, Nature 1999, 397:157-160), and ketamine (Sleigh et al. Trends Anaesthesia Critical Care 2014, 4:76-81). Calcium antagonists such as nimodipine also protect RGCs (e.g. Grosskreutz et al., Curr Eye Res 1999, 18:363-367). At least 2 other non-NMDA excitatory amino acid receptors are widespread in the retina and are likely involved in signal transmission between photoreceptor or bipolar cells and ganglion cells: the kainate receptor and the 2-amino-3-hydroxy-5-methyl-4-isoxazolepropionic acid (AMPA) receptor (DeVries & Schwartz, Nature 1999, 397:157-160). Inhibitors of these non-NMDA excitatory amino acid receptors, e.g. cis-2-3-piperidine dicarboxylic acid (cis-PDA), exert retinal neuroprotective effects (Weber et al., Graefes Arch Clin Exp Ophthalmol 1995, 233:360-365). Other inhibitors of kainate and AMPA receptors include 6-cyano-7-nitroquinoxaline-2,3-dione (CNQX), and examples of selective AMPA receptor antagonists are the 2,3-benzodiazepine compounds GYKI52466 and GYKI53655 (Paternain et al., Neuron 1995, 14:185-189).

Combination of NMDA- and non-NMDA-receptor antagonists may increase the protection against retinal neurodegeneration (Mosinger et al., Exp Neurol 1991, 113:10-17).

Other neuroprotective factors include insulin, neuroprotectin D1, brain-derived neurotrophic factor (BDNF), glial cell line derived neurotrophic factor (GDNF), ciliary neurotrophic factor (CNTF), nerve growth factor (NGF), adrenomedullin (AM), pigment epithelium-derived factor (PEDF), somatostatin (SST), interstitial retinol-binding protein (IRBP) (Simo & Hernandez, Trends Endocrinol Metabol 2014, 25:23-33).

In view of their action, any of the above exemplified compounds (non-exhaustive list) is capable of protecting neuronal cells, in particular retinal neuronal cells, to some extent, the whole group therefore in the current context being defined as neuroprotective compounds, in particular retinal neuroprotective compounds.

As already indicated, the invention relates to monospecific placental growth factor (PlGF) antagonists for use in treating, preventing, or delaying progression of ocular posterior fibrosis in a subject. Alternatively, the monospecific placental growth factor (PlGF) antagonist is for use in treating, preventing, or delaying progression of ocular posterior fibrosis without inducing ocular posterior neurodegeneration in a subject. As a further embodiment the monospecific placental growth factor (PlGF) antagonist for the above uses envisages further treating, preventing, or delaying progression of ocular posterior inflammation and/or ocular posterior neovascularization and/or vessel leakage.

The invention also relates to monospecific placental growth factor (PlGF) antagonists for use in maintaining or improving the visual acuity of a subject with an eye of which the retina is damaged.

It is clear that, in the above, the monospecific placental growth factor (PlGF) antagonist alone can be administered to an eye, i.e. without administering another compound different from the monospecific PlGF antagonist.

Alternatively, the monospecific placental growth factor (PlGF) antagonist may be administered to an eye after wash out of a vascular endothelial growth factor (VEGF) antagonist or a VEGF-receptor (VEGFR) antagonist previously administered to the same eye.

In a further alternative, a vascular endothelial growth factor (VEGF) antagonist or a VEGF-receptor (VEGFR) antagonist is administered to an eye after wash out of the monospecific placental growth factor (PlGF) antagonist previously administered to the same eye.

Further alternatives are envisaged including the combined administration of a monospecific placental growth factor (PlGF) antagonist and a second active compound. As such, in any of the above-described uses, the monospecific placental growth factor (PlGF) antagonist may be administered to an eye in combination with a second active compound wherein said second active compound is different from a vascular endothelial growth factor (VEGF) antagonist and different from a VEGF-receptor (VEGFR) antagonist.

Alternatively, the monospecific placental growth factor (PlGF) antagonist is administered to an eye in combination with a second active compound wherein said second active compound is different from a vascular endothelial growth factor (VEGF) antagonist and different from a VEGF-receptor (VEGFR) antagonist; and wherein said administration is after wash out of a vascular endothelial growth factor (VEGF) antagonist or VEGF-receptor (VEGFR) antagonist previously administered to the same eye.

In a further alternative, a vascular endothelial growth factor (VEGF) antagonist or a VEGF-receptor (VEGFR) antagonist is administered to an eye after wash out of the monospecific placental growth factor (PlGF) antagonist previously administered to the same eye in combination with a second active compound wherein said second active compound is different from a vascular endothelial growth factor (VEGF) antagonist and different from a VEGF-receptor (VEGFR) antagonist.

When a monospecific placental growth factor (PlGF) antagonist is combined with a second active compound, both can be administered to the eye each in a separate composition (each in the same or in a different pharmaceutically acceptable formulation). The second active agent can be administered prior to, concurrent with, or after the administration of the monospecific placental growth factor (PlGF) antagonist. Alternatively, both can be administered to the eye combined in a single composition (in the same pharmaceutically acceptable formulation).

Combinations of PlGF antagonist (with or without a further second active compound) and VEGF- or VEGFR-antagonist as described above can take many forms. For instance, administration of PlGF antagonist at the one hand and of VEGF- or VEGFR-antagonist at the other hand could be alternated (starting with either one in a first administration). Alternatively, a first administration of PlGF-antagonist, or of VEGF- or VEGFR-antagonist, respectively, could be followed by multiple subsequent administrations of VEGF- or VEGFR-antagonist, or of PlGF antagonist, respectively. In a further alternative, a first and second administration of PlGF-antagonist, or of VEGF- or VEGFR-antagonist, respectively, could be separated by multiple subsequent administrations of VEGF- or VEGFR-antagonist, or of PlGF antagonist, respectively.

Second active compounds in this context may be one active compound or a combination of more than one active compound. In particular, but not limiting, such second active compound may be an anti-inflammatory compound, an anti-angiogenic compound, an anti-fibrotic compound, an AGE-inhibiting compound, an ALE-inhibiting compound, an AGE-breaking compound, a carbonic anhydrase inhibitor, an NMDA-receptor antagonist, a kainate receptor antagonist, an AMPA-receptor antagonist, a neuroprotective agent, an agent for controlling the intra-ocular pressure, an anti-apoptotic agent, an antiviral compound, an antibiotic compound, an antifungal compound, an antihistamine, an anticoagulant, a thrombolytic compound, an anti-mitotic agent, an anesthetic agent, and agent inducing mydriasis, an agent inducing cycloplegia, an agent inducing posterior vitreous detachment (complete or incomplete), an agent inducing vitreous liquefaction, an integrin inhibitor, an anti-edema agent.

In view of the state of the art, any use of the monospecific placental growth factor (PlGF) antagonist as hereinabove described may of course be combined with photodynamic therapy, laser photocoagulation, radiation therapy or vitreal surgery.

Any use of the monospecific placental growth factor (PlGF) antagonist as hereinabove described may be characterized further in that the visual acuity of the subject is stabilized or improved (see “treatment/treating” for explanation of e.g. amelioration=improvement).

Any of the above can also be redrafted as methods for treating, preventing, or delaying progression of ocular posterior fibrosis in a subject. In particular the subject is a mammal, more in particular a human.

“Administering” means any mode of contacting that results in interaction between an agent (e.g. monospecific PlGF antagonist) or composition comprising the agent (such as a medicament) and an object (cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent, composition or medicament to the object.

The term “effective amount” refers to the dosing regimen of the agent (e.g. monospecific PlGF antagonist) or composition comprising the agent (e.g. medicament). The effective amount will generally depend on and will need adjustment to the mode of contacting or administration. The effective amount of the agent, composition or medicament, more particular its active ingredient, is the amount required to obtain the desired clinical outcome or therapeutic or prophylactic effect without causing significant or unnecessary toxic effects. To obtain or maintain the effective amount, the agent, composition or medicament may be administered as a single dose or in multiple doses. The effective amount may further vary depending on the severity of the condition that needs to be treated or the expected severity of the condition that needs to be prevented or treated; this may depend on the overall health and physical condition of the patient and usually the treating doctor's or physician's assessment will be required to establish what is the effective amount. The effective amount may further be obtained by a combination of different types of contacting or administration. In the context of the present invention the effective amount may more particularly be obtained by either one or more of administration of topical eye drops, administration by injection into the anterior chamber of an eye, administration by subconjunctival injection, administration by intravitreal injection, systemic administration, sustained- or slow-release administration (e.g. re-fillable eye implant, container with recombinant cells expressing the agent, erodible gel implant loaded with the agent, gene therapeutic modalities). Administration of a monospecific PlGF antagonist (with or without administration of a second active agent) by means of ocular injection typically is kept to a minimum, i.e., the frequency of repeat injections is kept to a minimum and can be adjusted to the further course of the eye disease or disorder, or any single symptom thereof.

The wash out period in the current context is the period during which an agent administered to the eye is washed out from the eye, e.g. due to clearing from the eye (e.g. into the systemic circulation or into tear fluid) or due to intraocular degradation or intraocular neutralization. In practice, the wash out period, i.e. the number of wash out hours or days, is the period during which no therapy is delivered or at the end of which the concentration of the active compound has decreased to or below the effective concentration. Alternatively, the wash out period is the period between two deliveries of therapeutic agents that can be the same or can be different. The wash out period will usually depend on the nature and dosing of the agent, i.e., by its pharmacokinetic properties, which are determined during the (pre-)clinical development of a potential new drug. Specifically in case of ocular drug administration by injection, the wash out period will preferably be long enough to avoid a high frequency of repeat injections.

An “agent for controlling the intra-ocular pressure” is an agent that stabilizes or lowers the intra-ocular pressure. Such medicaments include adrenergic blocking agents (beta blockers or sympatholytic drugs such as betaxolol, carteolol, levobunolol, metipanolol and timolol), adrenergic stimulating agents (sympathomimetic drugs such as aproclonidine, epinephrine, hydroxyamphetamine, phenylephrine, naphazoline and tetrahydrozaline), carbonic anhydrase inhibitors (such as systemic acetozolamide, and topical brinzolamide and dorzolamide), miotics (cholinergic stimulating agents, parasympathomimetic drugs such as carbachol and pilocarpine), osmotic agents (such as glycerin and mannitol), prostaglandin and prostaglandin analogues (prostamides, bimatoprost, unoprostone isopropyl, travoprost, latanoprost, natural prostaglandin, prostaglandin F2α, and FP prostanoid receptor agonists). When such medicaments are not efficient (or not anymore), then glaucoma filtration surgery is a viable treatment.

“Anticoagulants” include hirudins, heparins, coumarins, low-molecular weight heparin, thrombin inhibitors, platelet inhibitors, platelet aggregation inhibitors, coagulation factor inhibitors, anti-fibrin antibodies and factor VIII-inhibitors (such as those described in WO 01/04269 and WO 2005/016455).

“Thrombolytic agents” include urokinase, streptokinase, tissue-type plasminogen activator (tPA), urokinase-type plasminogen activator (uPA) and staphylokinase or any variant or derivative of any thereof such as APSAC (anisoylated plasminogen streptokinase activator complex), alteplase, reteplase, tenecteplase, and scuPA (single chain uPA), plasmin or any truncated variant thereof such as midiplasmin, miniplasmin, deltaplasmin and microplasmin.

“Anti-inflammatory agents” include steroids (e.g. predniso lone, methylpredniso lone, cortisone, hydrocortisone, prednisone, triamcinolone, dexamethasone) and non-steroidal anti-inflammatory agents (NSAIDs; e.g. acetaminophren, ibuprofen, aspirin), see also agents described higher.

“Antiviral agents” include trifluridine, vidarabine, acyclovir, valacyclovir, famciclovir, and doxuridine.

“Antibacterial agents” or antibiotics include ampicillin, penicillin, tetracycline, oxytetracycline, framycetin, gatifloxacin, gentamicin, tobramycin, bacitracin, neomycin and polymyxin.

“Anti-mycotic/fungistatic/antifungal agents” include fluconazole, amphotericin, clotrimazole, econazole, itraconazole, miconazole, 5-fluorocytosine, ketoconazole and natamycin.

“Anti-angiogenic agents” include agents described higher as well as, mini-trypthophanyl-tRNA synthetase (TrpRS) (Wakasugi et al., Proc Natl Acad Sci USA 2002, 99:173-177), anecortave acetate, combrestatin A4 prodrug, AdPEDF (adenovector capable of expressing pigment epithelium-derived factor), inhibitors of TGF-β, Sirolimus (rapamycin), endostatin, and possibly integrin inhibitors (U.S. Pat. No. 9,018,352).

“Anti-mitotic agents” include mitomycin C and 5-fluorouracyl.

“Antihistamine” includes ketitofen fumarate and pheniramine maleate.

“Anesthetics” include benzocaine, butamben, dibucaine, lidocaine, oxybuprocaine, pramoxine, proparacaine, proxymetacaine, tetracaine and amethocaine.

Other adjunct agents or drugs that can be used in conjunction with the monospecific PlGF antagonist include scopoloamine, atropine or tropicamide, to induce mydriasis (pupillary dilation) and/or cycloplegia (paralysis of the eye focusing muscle).

“Anti-edema agents” include inhibitors of plasma kallikrein (e.g. ecallantide; and KVD001, in phase I for treating DME, KalVista Pharmaceuticals; see WO 2014/006414) and some anti-inflammatory agents (see higher).

Whereas vitreous liquefaction seems a prerequisite for the induction of posterior vitreous detachment (PVD), liquefaction in itself does not lead to PVD, which was established by intravitreal administration of hyaluronidase. This protease is able to induce vitreous liquefaction but fails to produce a separation of the posterior vitreous from the inner limiting membrane (PVD) (Sebag et al., Trans Am Ophthalmol Soc 2005, 103:473-494; Williams, Ophthalmology 2008, 108:1902-1905; Lopez-Lopez et al., Curr Diabetes Rev 2009, 5:57-62). It has been demonstrated that combined ocular injection of chondroitinase ABC and MMP-3 can lead to PVD in the rabbit eye. Yet, this study also indicated that intravitreal injection of chondroitinase ABC together with MMP-3 resulted in liquefaction in all treated eyes (100%), while only 62.5% injected eyes exhibited PVD, indicating that 37.5% of the experimental eyes displayed only vitreous liquefaction and no PVD (Meng & Zeng, Zhonghua Yan Ke Za Zhi 2004, 40:625-631).

Several enzymes including plasmin, collagenase, hyaluronidase, dispase, chondroitinase, urokinase and nattokinase have been analyzed for their potential to induce pharmacologic vitreolysis. It has been demonstrated that plasmin and its truncated form microplasmin have the capacity to induce PVD in animal models as well as post-mortem human eyes (U.S. Pat. No. 5,304,118; GB2393121; WO2004/052228; Stalmans et al., New Engl J Med 2012, 367:606-615). Ocriplasmin (Jetrea®, ThromboGenics NV) is indeed the first approved drug that can be used as a non-chirurgical treatment for focal symptomatic vitreomacular adhesion (sVMA). Other, non-enzymatic, agents inducing PVD include urea and urea derivatives (e.g. WO 00/51620), and integrin inhibitors (e.g. U.S. Pat. No. 9,018,352).

The vitreous humor is a clear gel that occupies the space between the lens and the retina and it helps the eye to maintain its round shape. The vitreous gel consists mainly out of water molecules and only 1% macromolecules such as collagen, hyaluronic acid, and glycoproteins. These macromolecules form a network and establish a stable gel-like structure. Normal adhesion at the vitreo-retinal interface is mediated by interactions between the posterior vitreous cortex and the inner limiting membrane of the retina (Sebag et al. 2005, Trans Am Ophthalmol Soc 103:473-494). The inner limiting membrane mainly consists of collagen, fibronectin and laminin. Vitreo-retinal diseases comprise eye disorders, which can cause vision loss, due to aberrant interactions between the inner limiting membrane and the vitreous gel/posterior vitreous cortex. Such aberrant interactions often induce retinal damage, in turn inducing posterior ocular fibrotic responses. Anomalies at the vitreo-retinal interface can lead to permanent loss of vision and lead to symptoms or diseases such as partial posterior vitreous detachment, retinal tear, retinal detachment, symptomatic vitreomacular adhesion/traction, macular hole, idiopathic and secondary epiretinal membrane, proliferative vitreo-retinopathy, proliferative diabetic retinopathy, diabetic macular edema, cystoid macular edema, and age-related macular degeneration. The abnormal mechanical traction of the vitreous on the retina is presumed to be the underlying factor in many eye/ocular/retinal diseases and maculopathies (Skeie & Mahajan, PLOS One 2013, 8:e82140; Shao & Wei, Chin Med J 2014, 127:1566-1571). Depending on the traction site of the vitreous on the retina, different effects may emerge. Pulling on blood vessels may cause retinal and vitreous hemorrhage and may stimulate retinal neovascularization. Traction in the macular area may cause vitreo-macular traction syndrome, macular pucker, macular holes, and/or diabetic macular edema. If the traction site is in the periphery, retinal tears and/or retinal detachments may occur. If the optic disc is affected by anomalous traction of the vitreous, vitreo-papillary traction syndrome and aggravation of neovascularization of the optic disc, proliferative diabetic vitreoretinopathy and/or central retinal vein occlusion may result (Sebag, Graefe's Arch Clin Exp Ophthalmol 2004, 242:690-698).

Symptomatic vitreomacular adhesion (sVMA) is an eye condition in which the vitreous gel has an aberrantly strong adhesion to the retina. Over time, the gel tends to pull forward and this can cause retinal distortions resulting in visual deficits (i.e. metamorphopsia). In more advanced stages, sVMA (sometimes referred to as vitreomacular traction, VMT) can even cause a focal retinal tear or macular hole, which can lead to blindness. Typical for a VMT-associated macular hole is that the retina is not interrupted over its full-thickness (in contrast to full-thickness macular hole wherein all retinal layers are interrupted). Vitreous traction can be treated by means of a surgical intervention known as vitrectomy. Surgical vitrectomy is a standard treatment for sVMA, but this mechanical procedure to relieve vitreous traction remains critical and carries the high risk of damage to the retina. For this reason several proteases have been tested as an adjunct to vitrectomy or even to replace vitrectomy and/or for induction of pharmacological vitreolysis or pharmacological posterior vitreous detachment (PVD). Molecular therapy has the potential to improve visual outcomes and overcome the risks associated with surgical/mechanical vitrectomy. Several enzymes such as plasmin, collagenase, hyaluronidase, dispase, chondroitinase, urokinase and nattokinase have been analyzed for their potential to induce pharmacologic vitreolysis. As discussed above, ocriplasmin (a truncated variant of plasmin) has recently been registered and is currently the only available product capable of inducing PVD and capable of promoting macular hole closure. A possible synonym for VMA is anomalous PVD, defined as a partial PVD concurrent with persisting attachment of vitreous to the retina in the macular region. The attachment is of anomalous strength and can result in deformation of the retina. The partial PVD more particular occurs in the perifoveal area. VMA becomes symptomatic VMA (sVMA) when associated with any symptom(s) and/or disease(s). Focal or broad (s)VMA may occur, over a distance of less than or equal to 1500 μm or of over 1500 μm, respectively. More details on definitions and classifications can be found in Duker et al. (Ophthalmology 2013, 120:2611-2619). The current standard technology for determining the presence of (partial of full) PVD or (s)VMA is optical coherence tomography (OCT).

Vitrectomy, vitreolysis, vitreous liquefaction and/or induction of PVD is of benefit for a number of eye conditions such as vitreous floaters (motile debris/deposits of vitreous within the normally transparent vitreous humour which can impair vision), retinal detachment (a blinding condition which may be caused by e.g. vitreal fraction), macular pucker (scar tissue on macula; macula is required for sharp, central vision; macular pucker is also known as epi- or preretinal membrane, cellophane maculopathy, retina wrinkle, surface wrinkling retinopathy, premacular fibrosis, or internal limiting membrane disease), diabetic retinopathy (proliferative or non-proliferative) which may result in vitreal hemorrhage and/or formation of fibrous scar tissue on the retina (which may cause retinal detachment), macular holes (hole in macula causing a blind spot and caused by vitreal traction, injury or a traumatic event; can be full-thickness or not), vitreous hemorrhage (caused by diabetic retinopathy, injuries, retinal detachment or retinal tears, subarachnoidal bleedings (Terson syndrome), or blocked vessels), subhyaloid hemorrhage (bleeding under the hyaloid membrane enveloping the vitreous), macular edema (deposition of fluid and/or protein on or under the macula of the eye) and macular degeneration (starting with the formation of drusen; occurs in dry and wet form; if correlated with age coined age-related macular degeneration).

Full thickness macular holes are categorized as small (less than or equal to 250 μm), medium (over 250 μm but less than or equal to 400 μm) or large (over 400 μm) (Duker et al., Ophthalmology 2013, 120:2611-2619).

Age-related macular degeneration (AMD) and diabetic retinopathy (DR) patients can suffer from additional vitreo-retinal complications such as partially detached vitreous with traction, epiretinal membrane, tractional retinal detachment or macular hole for which PVD or sVMA resolution or VMT resolution could be beneficial. AMD and DR are both multifactorial eye disorders and ischemic damage plays a major role in their pathophysiology. Surgical and enzymatic PVD (or sVMA resolution or VMT resolution) seems to have a protective role against hypoxia-induced complications in AMD and DR, as PVD is associated with increased vitreal and retinal oxygenation. Moreover, it has been described that an aberrantly strong attached vitreous and/or vitreomacular traction is correlated with an increased risk of progressing to exudative AMD and proliferative DR (Williams et al., Ophthalmology 2001, 108:1902-1905; Haller et al., Ophthalmology 2010, 117:1087-1093; Roller et al., Ophthalmology 2010, 117:1381-1386). Furthermore, vitreo-retinal traction is a major pathological cause of visual deficits in DR, since it can induce diabetic macular edema. It has indeed been observed that release of mechanical traction on the retina by means of PVD can lead to reduction in diabetic macular edema (Williams et al., Ophthalmology 2001, 108:1902-1905; Haller et al., Ophthalmology 2010, 117:1087-1093).

The scientific literature indicates that surgical vitrectomy is successful in the treatment of a plethora of ocular diseases and disorders. Exemplary references are included hereafter in support of the wide therapeutical applicability of vitrectomy, including pharmacological vitrectomy. Ondes et al. (Jpn J Ophthalmol 2000, 44:91-93) discusses that the frequency of PVD is less in eyes with age-related macular degeneration (AMD) than in normal eyes and proposes a mechanism by which vitreoretinal adherence (i.e., the absence of PVD) negatively influences AMD. Stefansson et al. (Invest Ophthalmol Vis Sci 1990, 31:284-289) discloses a possible mechanism for halting diabetic retinal neovascularization by vitrectomy, i.e. prevention of hypoxia. Hypoxia is a factor known to induce compensatory neovascularization as complication of vein occlusion (retinal or other). Stefansson et al. 1990 experimentally induced retinal vein occlusion and noticed that retinal oxygen deprivation is less severe in vitrectomized eyes. Preventing the neovascularization complication of retinal vein occlusion clearly is a method of treatment. Moreover, newly formed retinal vessels often are brittle and thereby prone to occlusion or rupture. Successful treatment of endophthalmitis by vitrectomy in combination with antibiotics is disclosed by e.g. Snip et al. (Am J Ophthalmol 1976, 82:699-704). Tachi et al. (Semin Ophthalmol 1998, 13:20-30) observed improvement of diabetic macular edema after spontaneous vitreous detachment or after vitrectomy. Controlling diabetic macular edema not responding to laser treatment was reported as well to benefit from vitreolysis (Lovestam-Adrian & Larsson, Int Ophthalmol 2005, 26:21-26). Resolution of cystoid macular edema as well as improvement of visual acuity was obtained after vitrectomy as described by e.g. Federman et al. (Ophthalmology 1980, 87:622-628). Vallat (Graefes Arch Clin Exp Ophthalmol 1986, 224:238-239) describes successful surgical treatment, by means of vitrectomy, of retinal detachment from macular hole caused by vitreous fraction. The impact of vitrectomy on proliferative diabetic retinopathy is discussed in e.g. Federman et al. (Ophthalmology 1979, 86:276-282), the impact being such that continuous regression of the disease was observed with time after vitrectomy. Similar conclusions were made for pharmacologic vitreolysis by Li et al. (Invest Ophthalmol Vis Sci 2013, 54: 4964-4970). Hong et al. (Am J Ophthalmol 2001, 131:133-134) disclose the usefulness of vitrectomy in treating visually significant vitreous opacities that may develop as a complication of retinitis pigmentosa.

Pharmacologic vitreolysis, alone or as adjunct to surgical vitrectomy, is advocated to tackle the adverse effects of anomalous PVD, vitreous traction or VMT even early in eye diseases such as to prevent progression of the eye disease (Sebag, Graefe's Arch Clin Exp Ophthalmol 2004, 242:690-698).

A “pharmaceutically acceptable formulation” is, in the context of the current invention more particular an “ophthalmologically acceptable formulation”. A formulation in general is a composition comprising a carrier, diluent or adjunvant compatible with the one or more active ingredients to be formulated, the whole formulation being compatible with the intended use in the intended tissue or organ, etc. Examples of pharmaceutically acceptable formulations as well as methods for making them can be found, e.g., in Remington's Pharmaceutical Sciences (e.g. 20^{th} Edition; Lippincott, Williams & Wilkins, 2000) or in any Pharmacopeia handbook (e.g. US-, European- or International Pharmacopeia).

“Lubricants” include propylene glycerol, glycerin, carboxymethylcellulose, hydroxypropylmethylcellulose, soy lecithin, polyvinyl alcohol, white petrolatum, mineral oil, povidone, carbopol 980, polysorbate 80, dextran 70.

### EXAMPLES

### 1. Introduction

This study investigated the dose-response efficacy of PlGF inhibition by an anti-PlGF antibody (mouse PLGF-inhibiting antibody 5D11D4 or human PlGF-inhibiting antibody 16D3, ThromboGenics, Leuven, Belgium) on one or more of neovascularization, inflammation and collagen deposition in a mouse CNV model; and compared it to the effect of equimolar concentrations of an anti-VEGF-R2 antibody (DC101, produced by hybridoma cell line ATCC HB-11534), aflibercept (Eylea®, Bayer), triamcinolone acetonide (TAAC; Kenacort®, Bristol-Myers Squibb), and anti-murine VEGF antibody B20 (Liang et al. 2006, J Biol Chem 281:951-961). TAAC was used as reference for inflammation and fibrosis. A treatment schedule of a single injection of 1 μL TAAC was selected based on the activity in mouse CNV model described by Takata et al. (Takata et al., Sci Rep 2015, 5:9898). The effects of the anti-PlGF antibody 5D11D4 and the anti-VEGF-R2 antibody DC101 on survival of retinal ganglion cells was investigated in naïve mice and in diabetic mice.

When referring hereinafter to e.g. injection of or with anti-PlGF (or 5D11D4 or 16D3) or of or with anti-VEGFR2 (or DC101), this is to be understood as injections of or with the above-mentioned anti-PlGF antibody 5D11D4 or anti-PlGF antibody 16D3, or of or with the above-mentioned anti-VEGF-R2 antibody DC101. Likewise, e.g. treated with anti-PlGF, with 5D11D4 or with 16D3 is to be understood as treated with the above-mentioned anti-PlGF antibody 5D11D4 or with the above-mentioned anti-PlGF antibody 16D3.

### 2. Materials and Methods

All experimental animal procedures were approved by the Institutional Animal Care and Research Advisory Committee of the KU Leuven, according to the 2010/63/EU Directive. All animal procedures were performed in accordance with the ARVO Statement for the Use of Animals in Ophthalmic and Vision Research.

2.1. Mouse CNV Model

Mice (C57BL/6J, male, 8-10 weeks old) were anesthetized by intraperitoneal injection (135 μL) of a mixture of ketamine hydrochloride (Anesketin; 115 mg/L) and medetomidine (Domitor; 1 mg/mL) and their pupils dilated with one eye drop (50 μL) Tropicamide (Tropicol™; 5 mg/mL; Thea, Research Papers Clermont-Ferrand, France). Three laser burns were placed in one eye with a green laser at the 9, 12, 3 o'clock positions around the optic disk using a slit lamp delivery system with a hand-held cover slide as a contact lens moisturized with Genteal Gel™ (Novartis, Vilvoorde, Belgium). Each spot was placed with a spot size of 100 μm, laser duration of a 50 milliseconds and a power of 320 mW. Only spots, which showed bubble production, a sign of rupture of the Bruch's membrane and known to be necessary for triggering neovascularization, inflammation and fibrosis, were included. Finally, 300 μL atipamezole (Antisedan; 5 mg/mL) was injected intraperitoneally to reverse the effect of medetomidine and to reduce sedation time.

2.2. Intravitreal Administration

At different time points after lasering, the compounds were administered intravitreally (IVT). The animal was anesthetized with a mixture of ketamine hydrochloride/medetomidine and the eye was treated with a drop of 0.4% oxybuprocaine (Unicaine; Thea Pharma). Intravitreal injections (1 μL) to one eye according to Tables 1 to 3 were performed by using an analytic science syringe (SGE Analytic Science) and glass capillaries with a diameter of 50-70 μm at the end, controlled by the UMP3I Microsyringe Injector and Micro4 Controller (all from World Precision Instruments Inc., Hertfordshire, UK). Finally, atipamezole (Antisedan) was injected intraperitoneally to reverse the effect of medetomidine and to reduce sedation time.

2.3. Processing and Histology

On the day of sacrifice, mice were killed by cervical dislocation and the lasered eyes were enucleated and fixed in 1% (w/v) paraformaldehyde overnight. To analyze the in vivo efficacy of the different compounds, the retina was removed from the dissected posterior segments. These posterior eye cups, which included retinal pigment epithelium (RPE), the choroid and the sclera were stored in phosphate buffered saline (PBS).

2.3.1. Quantification of Inflammation

On day 5 after lasering, a rat anti-mouse CD45 and F4/80 antibody ( 1/100; Pharmingen, Erembodegem, Belgium) was used overnight to stain all leukocytes and macrophages, respectively, diluted in Tris-buffered saline (TBS)-Triton 0.3% (v/v). The following day, the tissues were incubated for 2 hours with rabbit anti-rat biotin labeled antibody ( 1/300; DakoCytomation A/S, Copenhagen, Denmark), diluted in TBS-Triton 0.3%. Antibody binding was visualized by fluorescent staining using streptavidin-Alexa-568 ( 1/200; Molecular Probes, Life Technologies, Eugene, Oreg., USA) in TBS-Triton 0.3% for 2 hours. The flatmounts of the posterior eye cups were mounted with Prolong Gold with 4′,6-diamidino-2-phenylindole (DAPI, Molecular Probes). Images were obtained using a microscope with a digital camera

TABLE 1
Study Design

| | | | | | | Endpoint | | | |
|---|---|---|---|---|---|---|---|---|---|
| | | | | Endpoint | | D7—neovascularization | | End point | |
| | | | | D5—inflammation | | and leakage | | D30—fibrosis | |
| Treatment | | | | Time point of | | Time point of | | Time point of | |
| Group | Treatment | Dose/1 μL | | IVT injection | Read-out | IVT injection | Read-out | IVT injection | Read-out |
| 1 | 5D11D4 | 3.1 | μg | D0 | CD45 and F4/80 | D0 | FITC + FA | D0, 4, 10 and 20 | Col1a |
| 2 | 5D11D4 | l.5 | μg | D0 | CD45 and F4/80 | D0 | FITC + FA | D0, 4, 10 and 20 | Col1a |
| 3 | 5D11D4 | 0.77 | μg | D0 | CD45 and F4/80 | D0 | FITC + FA | D0, 4, 10 and 20 | Col1a |
| 4 | IgG (1C8) | 3.1 | μg | D0 | CD45 and F4/80 | D0 | FITC + FA | D0, 4, 10 and 20 | Col1a |
| 5 | DC101 | 3.1 | μg | D0 | CD45 and F4/80 | D0 | FITC + FA | D0, 4, 10 and 20 | Col1a |
| 6 | aflibercept | 2.4 | μg | D0 | CD45 and F4/80 | D0 | FITC + FA | D0, 4, 10 and 20 | Col1a |
| 7 | aflibercept | 20 or | | D0 | CD45 and F4/80 | D0 | FITC + FA | D0, 4, 10 and 20 | Col1a |
| | | 40 μg | | | | | | | |
| 8 | TAAC | 40 | μg | D0 | CD45 and F4/80 | / | / | D0, 4, 10 and 20 | Col1a |
| 9 | TAAC | 4 | μg | D0 | CD45 and F4/80 | / | / | D0, 4, 10 and 20 | Col1a |

| | | | | | | | | |
|---|---|---|---|---|---|---|---|---|
| 10 | PBS | NA | D0 | CD45 and F4/80 | / | / | D0, 4, 10 and 20 | Col1a |
| 11 | aflibercept | NA | D0 | CD45 and F4/80 | / | / | D0, 4, 10 and 20 | Col1a |
| | buffer | | | | | | | |
| 12 | TAAC | NA | D0 | CD45 and F4/80 | / | / | D0, 4, 10 and 20 | Col1a |
| | buffer | | | | | | | |

IgG: irrelevant IgG antibody 1C8;
DC101: murine anti-murine VEGFR-2 antibody;
TAAC (Kenacort ® ): triamcinolone acetonide;
5D11D4: murine anti-murine P1GF antibody;
PBS: phosphate-buffered saline;
IVT: intravitreal;
Col1a: collagen 1a;
aflibercept: Eylea ® ;
D0, D4, D5, D7, D10, D20, D30: day 0, day 4, day 5, day 7, day 10, day 20, day 30, respectively;
FITC: fluorescein isothiocyanate conjugated dextran;
FA: fluorescein angiography.

(Axiocam MrC5; Carl Zeiss, Oberkochen, Germany) at a magnification of 20×. Morphometric analyses were performed using commercial software (Axiovision; Zeiss, Oberkochen, Germany). The density of inflammation was determined by calculating the CD45-positive area as a proportion of the total lesion.

2.3.2. Quantification of Neovascularization

On day 7 after lasering, angiogenesis was investigated using retrobulbar perfusion with 200 μL of fluorescein isothiocyanate (FITC)-conjugated dextran (50 mg/mL, Mr 2×10⁶ Da; Sigma-Aldrich, Diegem, Belgium) for 2 minutes. The flat mounts of the posterior eye cups were mounted with Prolong Gold with 4′,6-diamidino-2-phenylindole (DAPI, Molecular Probes). Images were obtained using a microscope with a digital camera (Axiocam MrC5; Carl Zeiss, Oberkochen, Germany) at a magnification factor of 20. Morphometric analyses were performed using commercial software (Axiovision; Zeiss, Oberkochen, Germany). The density of blood vessels was quantified by calculating the FITC-dextran-positive area as a proportion to the total CNV lesion area in the samples. Fluorescein angiography (FA) was performed on day 6 after laser to investigate vascular leakage.

2.3.3. Quantification of Collagen Deposition

To stain for the presence of collagen type 1 (Colla) protein in the laser spots at day 30 after laser, a rabbit anti-collagen antibody (Abcam, 1/270) was used overnight, diluted in Tris-buffered saline (TBS)-Triton 0.5% (v/v) at 4° C. The following day, the tissues were incubated for 2 hours with goat anti rabbit IgG Alexa Fluor 555 (Life Technologies; A-21428), diluted 1/100 in TBS-Triton 0.3% (v/v) at 4° C. The flat mounts of the posterior eye cups were mounted with Prolong Gold with 4′,6-diamidino-2-phenylindole (DAPI, Molecular Probes). Images were obtained using a microscope with a digital camera (Axiocam MrC5; Carl Zeiss, Oberkochen, Germany) at a magnification factor of 20. Morphometric analyses were performed using commercial software (Axiovision; Zeiss, Oberkochen, Germany). The density of collagen deposition was determined by calculating the Colla-positive area as a proportion of the total lesion.

2.4. Neuroretinal Safety

To investigate the safety of anti-PlGF and anti-VEGF-R2 on the retinal ganglion cell layer, naive mice (C75Bl/6 or Swiss mice) were injected with 25 mg/kg of anti-PlGF, anti-VEGF-R2 or control IgG 3 times a week for 6 weeks. Afterwards the mice were sacrificed and immunohistochemistry for the neuronal marker NeuN was performed. The samples were incubated overnight with the primary mouse anti-NeuN (Chemicon MAB377) 1/500. As secondary antibody, rabbit anti-mouse biotin-labeled 1/400 (Dako E0646) was added for 45 minutes. Subsequently, the sections were incubated with Streptavidin-HRP 1/100 in TNB for 30 minutes, followed by amplification with Biotin (kit NEL700) 1/50 in amplification buffer for 8 minutes and again incubated with Streptavidin-HRP 1/100 for 30 minutes (all from Perkin Elmer, Life Sciences). Hereafter, a 3,3-diaminobenzidine (DAB) staining (Fluka-Sigma Aldrich) was performed by adding peroxide to the tissue and a counterstaining was done with Harris hematoxylin (Merck). Viable RGCs were quantified 2 times on the same serial section on a defined length of the retina (250 μm) on either side of the optic nerve.

A TUNEL (terminal deoxynucleotidyl transferase-mediated biotinylated UTP nick end labeling) staining was performed to investigate apoptotic signal. Serial sections were deparaffinized, washed and treated with proteinase K ( 1/500; Qiagen, Venlo, the Netherlands) for 15 minutes. Next, sections are incubated with the TUNEL reaction mixture (In Situ Cell Death Detection Kit, POD; Roche, Mannheim, Germany) at 37° C. for 1 hour. Afterwards, the slides were mounted with Prolong Gold containing DAPI (Molecular Probes). Each section was scanned systematically from the temporal to the nasal or a serrata for fluorescent cells indicative of apoptosis. Positive labeled cells in the INL were counted.

2.5. Streptozotocin (STZ)-Induced Diabetic Mouse Model

C57BL/6J mice (male, 3-5 weeks old, Charles River) were rendered diabetic with five consecutive daily intraperitoneal injections of streptozotocin (STZ; Sigma Aldrich, St. Louis, Mo., USA), at 50 mg/kg. STZ was freshly dissolved (15-20 minutes prior use) in 6.6 mL Na-Citrate (CAM) buffer, yielding in a 7.5 mg/mL concentration at pH of 4.7. Control non-diabetic mice received five consecutive injections of CAM buffer alone. Development of diabetes was defined by blood glucose levels higher than 300 mg/dL and was monitored weekly after the first STZ or CAM injection by use of a glucose meter and strips (Glucomen, Menarini). Only animals with consistently elevated glucose levels for 3 weeks were used in the study. At 7 weeks after diabetes onset, mice of the different diabetes groups were randomized for the various treatment groups. Intraperitoneal injection of 10 times-diluted (60 mg/kg final dose) sodium pentobarbital (Nembutal, 60 mg/mL; CEVA, Sante Animale, Brussels, Belgium) was used to induce general anesthesia and the eye was treated with a drop of unicain 0.4% (Thea Pharma, France). Intravitreal injection(s) of 5D11D4 (5.4 μg, anti-PlGF antibody), DC101 (6.2 μg, anti-VEGFR-2 antibody) or PBS were administered in one eye (see Table 2). Eight weeks after diabetes onset (1 week after start IVT treatment), mice were sacrificed to investigate retinal ganglion cell (RGC) density. On the day of sacrifice, mice were killed by cervical dislocation and eyes were enucleated and fixed in 1% (w/v) paraformaldehyde overnight.

TABLE 2
Study Design Mouse STZ model

| Treatment | | | End point W8-RGC density | |
|---|---|---|---|---|
| Group | IVT injection | Dose | Time point of IVT injection | Read-out |
| 1 | no | NA | NA | Brn3a staining |
| 2 | PBS | NA | Week 7 after diabetes onset (every 2 days) | Brn3a staining |
| 3 | 5D11D4 | 5.4 μg/eye | Week 7 after diabetes onset (every 2 days) | Brn3a staining |
| 4 | DC101 | 6.2 μg/eye | Week 7 after diabetes onset | Brn3a staining |

DC101: murine anti-murine VEGFR-2 antibody;
5D11D4: murine anti-murine PlGF antibody;
PBS: phosphate-buffered saline;
IVT: intravitreal;
Brn3a: see 2.5.1;
RGC: retinal ganglion cell;
W8: week 8.

2.5.1. Quantification of RGC Density in Mouse STZ Model.

In the mouse STZ model, viable RGCs were visualized by Brn3a immunostaining at 8 weeks after diabetes onset (1 week after start IVT injections). Eyes were enucleated and placed in 1% (w/v) formaldehyde ON. Retinal sections (7 μm) of the injected eyes were stained with Brn3a mouse monoclonal antibody (dilution 1/100, MAB1585-Millipore) to visualize the viable RGCs. The Vector® M.O.M.™ Immunodetection Kit (BMK-2202) was used to process the retinal cross-sections for Brn3a immunoreactivity. BRN3A (POU4F1) is a class IV POU domain-containing transcription factor highly expressed in the developing sensory nervous system and in cells of the B- and T-lymphocytic lineages (Gerrero et al. 1993, Proc Natl Acad Sci USA 90:10841-10845) and is a reliable marker for retinal ganglion cells (Nadal-Nicolas et al. 2009, Invest Ophthalmol Vis Sci 50:3860-3868). Metamorph software (Leica, Wetzlar, Germany) was used to count viable RGCs. RGC density was measured by a masked reader in the central retina at two locations, on the anterior and posterior side of the optic nerve, based on the localization of the vascular leakage in this model. Images of 3 different serial sections containing the optic nerve head were used (six measurements in total). To quantify RGC density, ganglion cell nuclei were counted 250 μm form the optic nerve head on a defined length of the retina (250 μm on either side of the optic nerve head).

2.6. Pericyte Coverage and Posterior Fibrosis in Mouse CNV Model

The mouse CNV model as described in Example 2.1 was used. At different time points after lasering, compounds were administered intraperitoneally (IP), or intravitreally (IVT), as indicated in Table 3.

TABLE 3
Study Design Mouse CNV model

| | | | | Endpoint | | | |
|---|---|---|---|---|---|---|---|
| | | | | D14—pericyte | | End point | |
| | | | | coverage | | D30—fibrosis | |
| Treatment | | | | Time point of | | Time point of | |
| Group | Treatment | Dose | | IP injection | Read-out | IVT injection | Read-out |
| 1 | IgG | 25 | mg/kg | 3×/week | SMA staining | / | / |
| 2 | DC101 | 25 | mg/kg | 3×/week | SMA staining | / | / |
| 3 | 5D11D4 | 25 | mg/kg | 3×/week | SMA staining | / | / |
| 4 | PBS | NA | | / | / | D0, 4, 10 and 20 | Col1a staining |
| 5 | TAAC | 40 | μg/eye | / | / | D0, 4, 10 and 20 | Col1a staining |
| 6 | 5D11D4 | 3.1 | μg/eye | / | / | D0, 4, 10 and 20 | Col1a staining |
| 7 | 16D3 | 3.1 | μg/eye | / | / | D0, 4, 10 and 20 | Col1a staining |
| 8 | B20 | 3.1 | μg/eye | / | / | D0, 4, 10 and 20 | Col1a staining |
| 9 | aflibercept | 2.4 | μg/eye | | | D0, 4, 10 and 20 | Col1a staining |

IgG: irrelevant IgG antibody 1C8;
DC101: murine anti-murine VEGFR-2 antibody;
TAAC: triamcinolone acetonide;
5D11D4: murine anti-murine P1GF antibody;
16D3: murine anti-human P1GF antibody;
B20: murine anti-murine VEGF antibody;
PBS: phosphate-buffered saline;
IP: intraperitoneal;
IVT: intravitreal;
SMA: smooth muscle cell actin;
Col1a: collagen 1a;
D0, D4, D10, D14, D20, D30: day 0, day 4, day 10, day 14, day 20, day 30, respectively.

2.6.1. Quantification of Pericyte Coverage in Mouse CNV.

To study pericyte coverage as a marker of vessel maturation, smooth muscle cell actin (SMA) was examined on serial 7 mm paraffin sections. Immunohistochemistry was performed with rat anti-αSMA antibody (Sigma Aldrich) 1/500 diluted in tris natriumchloride blocking reagent (TNB) as primary antibody and rabbit-anti-mouse-HRP (horseradish peroxidase) labeled as secondary antibody for 45 minutes (Dako) 1/100 in TNB with pre-immune mouse serum 10% (v/v). The amount of SMA cells present in the lesion was determined by the use of commercial software (Axiovision; Zeiss, Oberkochen, Germany).

2.7. Alternating Anti-PlGF and Anti-VEGF in Mouse CNV Model

In a study design similar to that as depicted in Table 1 and Table 3, and for the purpose of determining the effect on collagen deposition/posterior ocular fibrosis, different dosing regimens of anti-PlGF antibody 5D11D4 and of anti-VEGF antibody B20 are compared. One such study design is given in Table 4. Group 1 of this study is expected to yield results similar to those obtained with aflibercept. This study is in part (group 2) initiating the determination of the washout period (from a murine eye), as well as initiating exploration of alternating anti-PlGF/anti-VEGF combinations as described higher herein.

TABLE 4
Study design mouse CNV model

| | | | | | | | number |
|---|---|---|---|---|---|---|---|
| | | | time point | | | | of |
| group | treatment | Dose/eye | D0 | D4 | D10 | D20 | animals |
| 1 | 5D11D4 + | 3.1 μg + | 5D11D4 + | 5D11D4 + | 5D11D4 + | 5D11D4 + | 6 |
| | B20 | 3.1 μg | B20 | B20 | B20 | B20 | |
| 2 | 5D11D4 | 3.1 μg or | 5D11D4 | B20 | 5D11D4 | B20 | 6 |
| | or B20 | 3.1 μg | | | | | |
| 3 | 5D11D4 | 3.1 μg or | B20 | 5D11D4 | 5D11D4 | 5D11D4 | 6 |
| | or B20 | 3.1 μg | | | | | |

2.8. Statistical Analysis

Comparisons between two experimental groups were performed using unpaired Student's t-tests. To compare more than two groups, one-way ANOVA analysis with treatment as variable was performed using Graph Pad Prism 5 with a Bonferroni post hoc analysis test.

A dataset of ‘Leuven Biostatistics and Statistical Bioinformatics Centre’ (L-BioStat) was used to determine statistical power. Power was given for an independent two-sided t-test with an alpha of 0.05 to detect a difference in means between two groups assuming equal variance and equal group size. Data are represented as mean±standard error of the mean (SEM), unless stated otherwise.

### 3. Results

To determine the therapeutic anti-angiogenic, anti-leakage anti-inflammatory and anti-anti-fibrotic potential of PlGF inhibition in a murine model of CNV, mice were treated with IVT or IP injections of 5D11D4, 16D3, DC101, B20, IgG, aflibercept, TAAC and their respective buffers (Table 1). All animals were clinically examined every other day and inflammation was investigated at day 5 after laser, neovascularization/leakage (including pericyte coverage) at day 7 or 14 and fibrosis was investigated at day 30 after laser. No treatment-related differences in pre- and post-treatment body weights at day 10, 20 and 30 were detected (data not shown).

3.1. Inflammation (Study Design: Table 1)

Analysis of F4/80 staining confirmed the previously published results (Van de Veire et al., Cell 2010, 141:178-190) that anti-PlGF was able to significantly decrease the infiltration of macrophages with 48%, whereas DC101 had no effect. Administration of aflibercept also reduced macrophage infiltration with 52%, as compared to IgG (P<0.05).

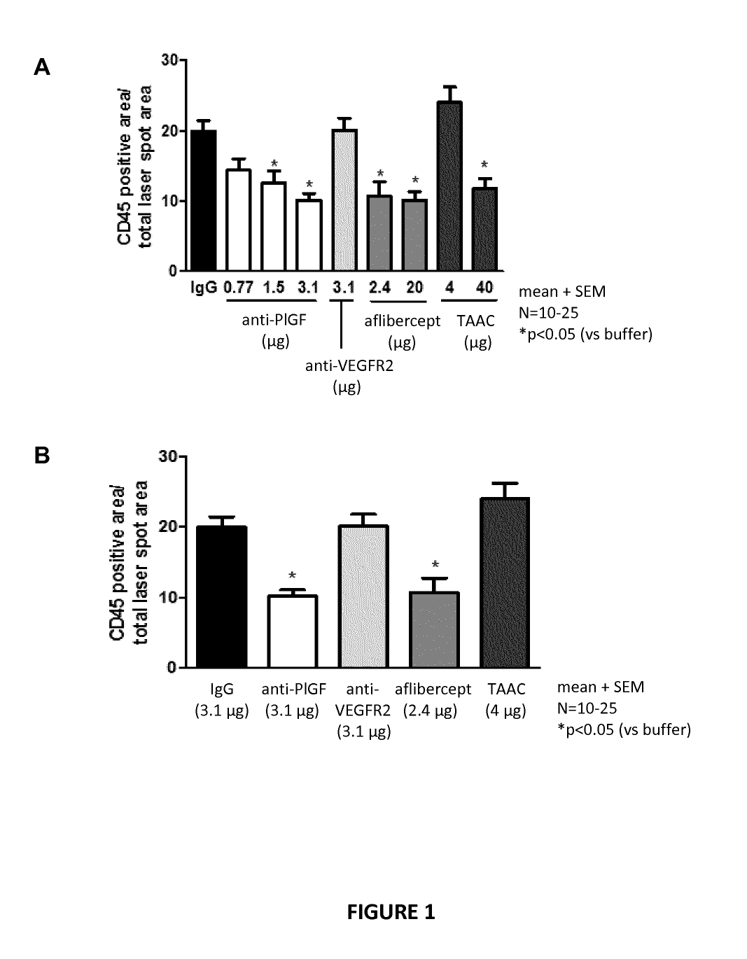

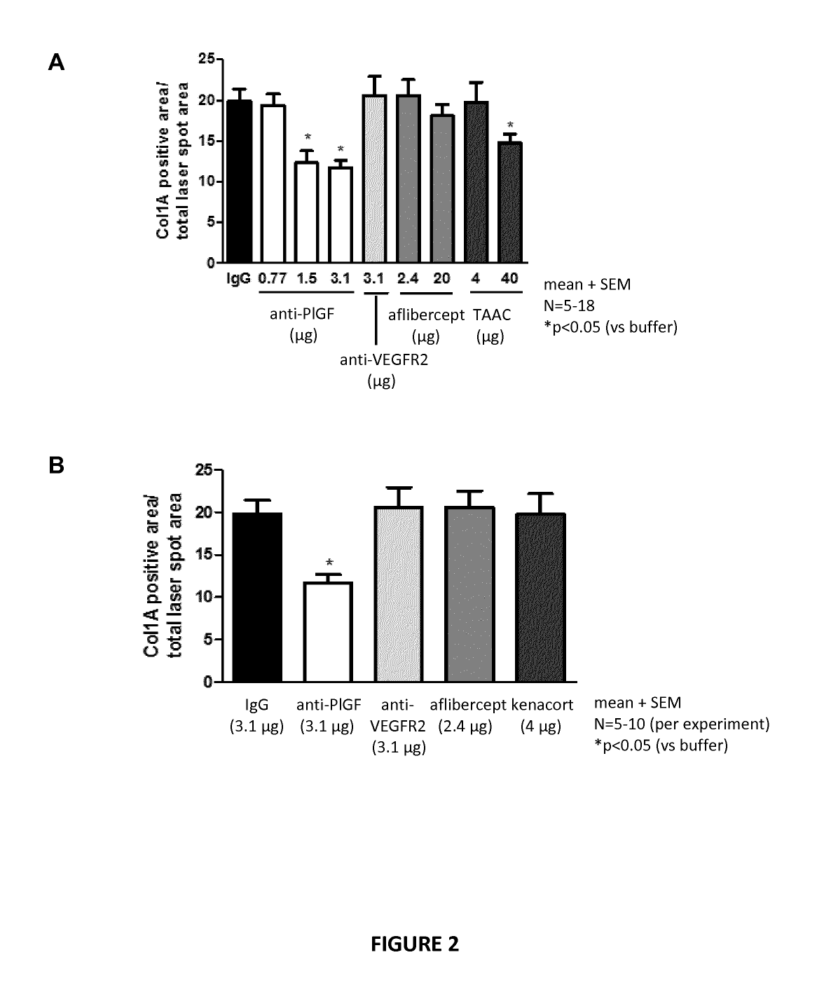

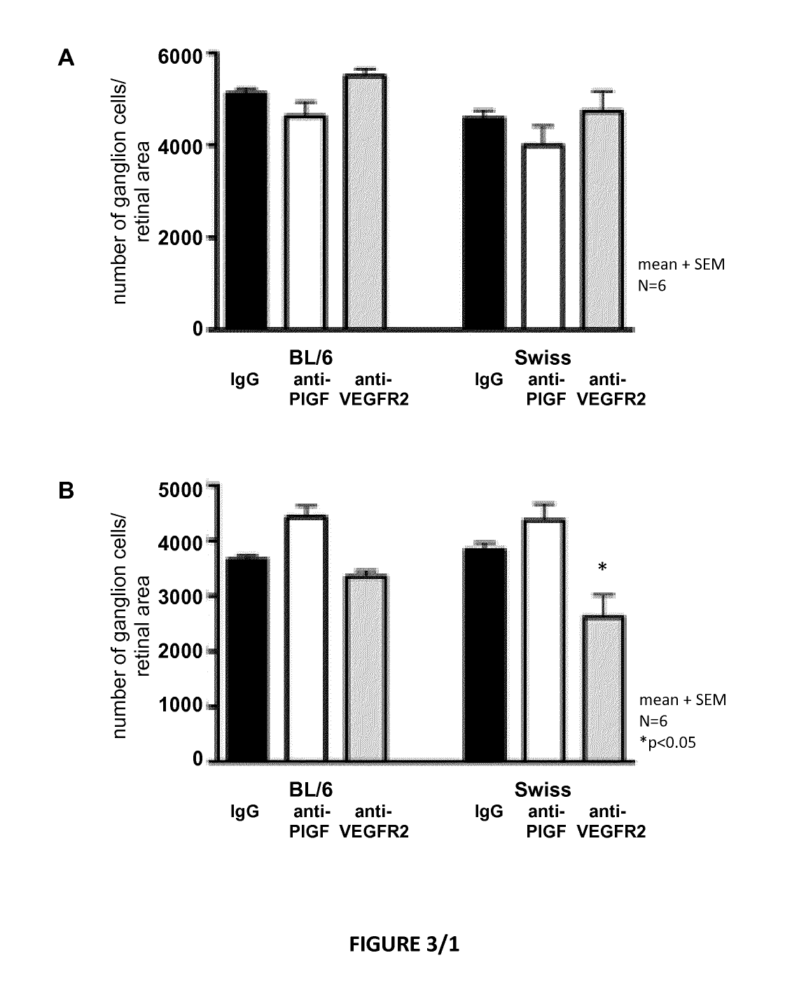

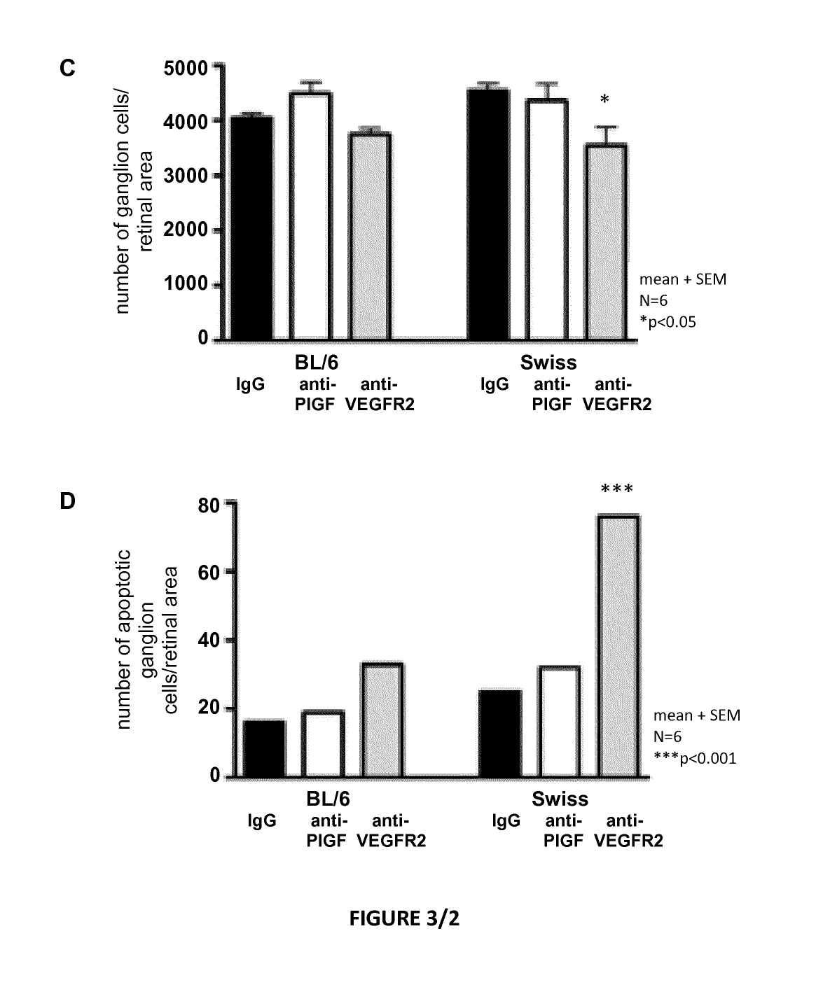

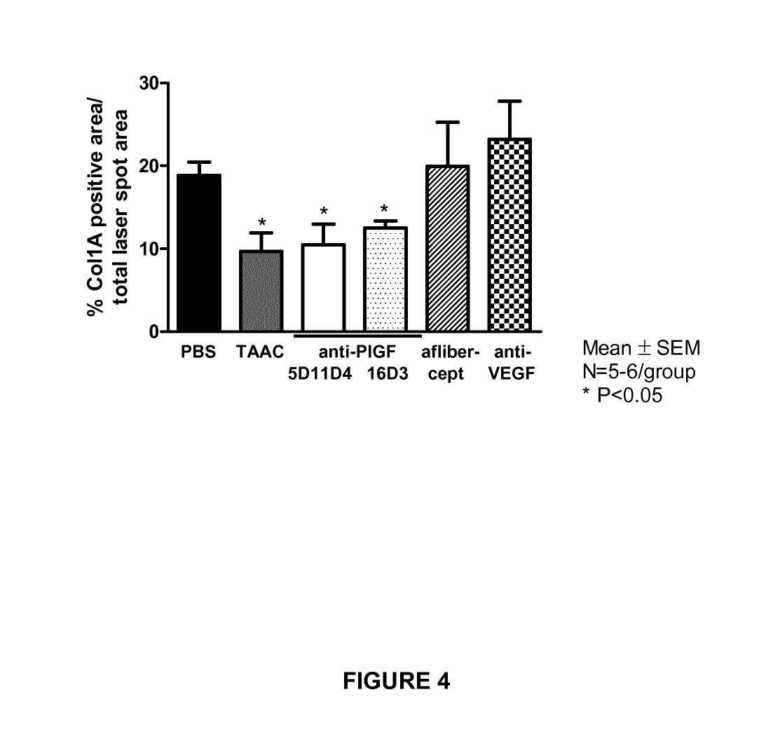

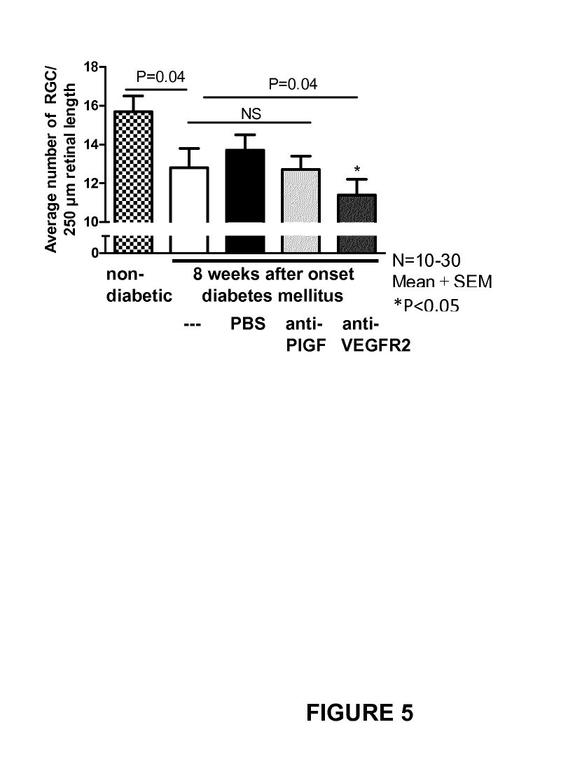

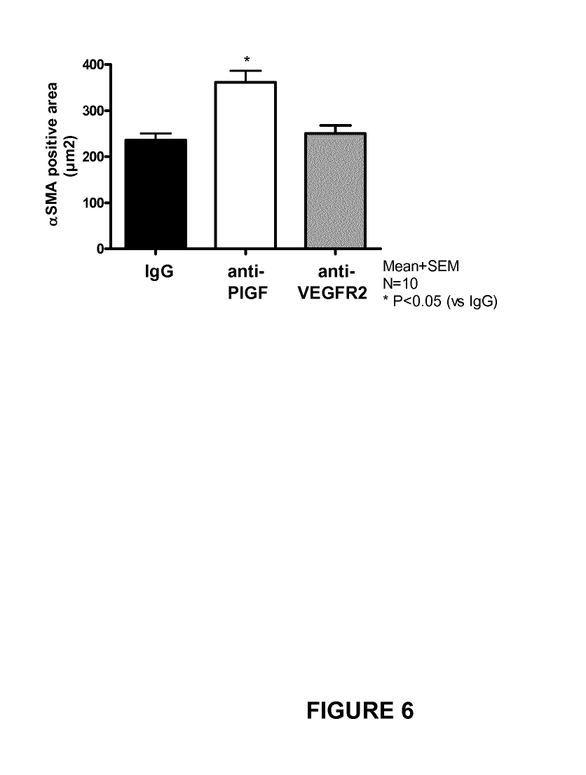

For the first time, the effect of leukocytes were investigated. Five days after lasering, CD45-immunohistochemical staining showed a dose-dependent significant reduction in leukocyte infiltration in the eyes of the group treated with the anti-PlGF antibody. Indeed, inflammation was significantly reduced with 36% and 46% of 1.5 μg and 3.1 μg 5D11D4, respectively (n=10-25; P<0.05 compared to IgG). This was comparable to 2.4 μg and 20 μg of aflibercept which induced a similar reduction of approximately 50%, respectively (P<0.05). Only the highest concentration of TAAC (40 μg) decreased the leukocyte infiltration (P<0.05), whereas lower concentrations did not have an effect. Importantly, a single administration of DC101 had no effect on leukocyte infiltration (FIG. 1A). After comparing the effect of equimolar concentrations of the different compounds, it can be concluded that both 5D11D4 and aflibercept reduce leukocyte infiltration with 50%, whereas DC101 and TAAC had no effect (FIG. 1B). Of note, TAAC only had an anti-inflammatory effect when administered at the highest dose of 40 μg. All the respective buffers, PBS, aflibercept- and TAAC-buffer were not different from IgG (P>0.05; data not shown).

3.2. Neovascularization and Leakage (Study Design: Table 1)

Previously published results were confirmed that inhibition of PlGF equally reduced neovascularization and leakage in the mouse CNV model, as compared to DC101 (Van de Veire et al., Cell 2010, 141:178-190). These effects were also comparable to those of aflibercept.

3.3. Collagen Deposition (Study Design: Tables 1 and 3)

Thirty days after lasering, collagen determination by immunohistochemical staining showed a dose-dependent and significant reduction in fibrosis in the eyes of the group treated with the anti-PlGF antibody. Indeed, collagen deposition was significantly reduced with 43% and 44% of 1.5 μg and 3.1 μg 5D11D4, respectively (n=5-18; P<0.05 compared to IgG). This was comparable to the highest concentration of TAAC (40 μg) whereas lower concentrations did not have an effect. In contrast, repeated administration of DC101 (3.1 μg) and aflibercept (2.4 μg and 20 μg) had no effect on collagen deposition (FIG. 2A). After comparing the effect of equimolar concentrations of the different compounds, it can be concluded that only 5D11D4 delays the process of wound healing, whereas aflibercept, DC101 and TAAC had no effect (FIG. 2B). Of note, TAAC only had an anti-fibrotic effect when administered at the highest dose of 40 μg. All the respective buffers, PBS, aflibercept- and TAAC-buffer were not different from IgG (P>0.05; data not shown).

In a repeat experiment, a further anti-PlGF antibody, 16D3 (anti-human PlGF), and an anti-VEGF-A antibody (B20), were included. Thirty days after lasering, collagen determination by immunohistochemical staining showed a significant reduction in fibrosis in the eyes of the group treated with both anti-PlGF antibodies. Indeed, collagen deposition was significantly reduced with 44% and 34% of 3.1 μg/eye 5D11D4 or 16D3, respectively (n=5-6; P<0.05 compared to PBS). This effect was comparable to the administration of the steroid, TAAC (40 μg/eye), showing a significant reduction of 49% in collagen deposition (P<0.05, FIG. 4). Administration of equimolar amounts of aflibercept (2.4 μg/eye) and the anti-VEGF antibody B20 (3.1 μg/eye) did not reduce fibrosis compared to PBS (P<0.05; FIG. 4).

3.4. Neuroretinal Safety/Naïve Mice

To study the effect of PlGF and VEGF-R2 inhibition on retinal ganglion cells (RGCs), C57Bl/6 mice were injected for 2, 4 and 6 weeks with 5D11D4, DC101 or isotype matched irrelevant control IgG (all three antibodies injected 3 times per week, intraperitoneally) and counted the number of RGCs on NeuN staining. The ganglion cell density (RGC/retinal area) was not significantly different between the 3 treatment groups at the three described time points: 5100 for control IgG versus 4600 for 5D11D4 and 5500 for DC101 (n=6; P=NS). A TUNEL staining confirmed that the number of apoptic cells per retinal area in the ganglion cell layer was comparable in the aPlGF versus control IgG treated mice after 6 weeks: 16±2 for control IgG versus 20±4 for 5D11D4. A trend for increase in apoptotic cells were present for the DC101 treated mice: 35±4 apoptotic cells per retinal area (n=6; P=0.10). Subsequently, these experiments were repeated in Swiss mice that carry the retinal degeneration gene mutation (Rd gene) and develop photoreceptor degeneration at the age of P19-24 (Caravaggio and Bonting, Exp Eye Res 1963, 2:12-19). Indeed, Swiss mice treated with DC101 exhibited an increased number of apoptotic cells with 33% in the ganglion cell layer on TUNEL staining (p<0.001) and a reduced RGC density of 32% after 4 weeks (n=6, p=0.05). In contrast anti-PlGF did not induce any alteration in RGC density or apoptotic rate (FIG. 3 A-D).

3.5. RGC Density in Mouse STZ Model (Study Design: Table 2)

To visualize the RGC density after compound administration, retinal sections were stained with a Brn3a mouse monoclonal antibody at 8 weeks after diabetes onset. The number of RGC in the central retina (250 μm form the optic nerve head on either sides of the optic nerve) of diabetic mice injected with STZ (no IVT) was significantly lower compared to non-diabetic mice (12.8±1.0/250 μm retinal length versus 15.7±0.8/250 μm retinal length, respectively, P=0.05; FIG. 5). RGC density after administration of 5D11D4 did not significantly differ from the PBS injected mice, whereas DC101 injection significantly reduced the RGC density with 20%, as compared to buffer (P<0.05, FIG. 5).

3.6. Pericyte Coverage in Mouse CNV Model (Study Design: Table 3)

Pericyte coverage was investigated at 14 days after lasering in the mouse CNV model. Analysis showed that the area of αSMA-positive vessels within the lesions was comparable between IgG-treated mice and mice injected with anti-VEGF-R2 antibody (25 mg/kg) (n=10, p>0.05), whereas an increase of 35% in pericyte-covered vessels was present in the anti-PlGF-treated group. This means that anti-PlGF treatment (25 mg/kg) improved the maturation of choroidal blood vessels (n=10, p<0.05; FIG. 6).

### 4. Conclusions

It can be concluded that PlGF-inhibiting or -neutralizing antibodies are able to reduce fibrosis, as well as reducing neovascularization, leakage, and inflammation, all of this without affecting RGC survival.

Of these, the effect on fibrosis is unique to the monospecific PlGF antagonist as not shared by VEGF-inhibitors (VEGF- and VEGFR2-inhibitors) and dual VEGF-/PlGF-inhibitors. Of importance, fibrosis was studied at day 30 after lasering, i.e. at a time point at which the collagen deposition seems to slow down (Van Bergen et al., Invest Ophthalmol Vis Sci 2015, 56:5280-5289).

The absence of neurodegenerative properties of the monospecific PlGF antagonist is further remarkable as also not shared by VEGF inhibitors. The effect of monospecific PlGF antagonists on RGC was so far unknown. Izawa et al. (Invest Ophthalmol Vis Sci 2015, 56:6914-6924) previously reported protective effects of an anti-PlGF antibody against light-induced photoreceptor degeneration in an experimental model resembling dry age-related macular degeneration (dry AMD). This property of anti-PlGF antibodies is shared with anti-VEGF antibodies (Cachafeiro et al., Cell Death Dis 2013, 4:e781). In contrast with the current results, Inoue et al. (J Neurosci Res 2014, 92:329-337) reported a neuroprotective effect exerted by PlGF itself, which seems negated by anti-PlGF. A possible explanation for this is that Inoue et al. used in vitro cultured cells, which is different from the current study performed in vivo. The dual-specific VEGF/PlGF inhibitor aflibercept was also reported to increase retinal pigment epithelium (RPE) cell death (Julien et al., Br J Ophthalmol 2014, 98:813-825).

In view of the above, monospecific PlGF antagonists differentiate themselves from VEGF inhibitors which are currently the gold standard therapies in clinical practice.

1. A method of treating, preventing, or delaying progression of ocular posterior fibrosis in a subject, the method comprising administering an effective amount of a monospecific placental growth factor (PlGF) antagonist to the subject.

2. A method of treating, preventing, or delaying progression of ocular posterior fibrosis in a subject, the method comprising administering an effective amount of a monospecific placental growth factor (PlGF) antagonist to the subject, wherein ocular posterior neurodegeneration is not induced in the subject.

3. The method of claim 1, wherein the method further treats, prevents, or delays progression of ocular posterior inflammation and/or ocular posterior neovascularization and/or vessel leakage.

4. The method of claim 1, wherein the method further maintains or improves the visual acuity of a subject with an eye of which the retina is damaged.

5. The method of claim 1, wherein the monospecific placental growth factor (PlGF) antagonist alone is administered to an eye.

6. The method of claim 1, wherein the administering of the monospecific placental growth factor (PlGF) antagonist is to an eye after wash out of a vascular endothelial growth factor (VEGF) antagonist or a VEGF-receptor (VEGFR) antagonist previously administered to the same eye.

7. The method of claim 1, wherein the monospecific placental growth factor (PlGF) is administered to an eye and further comprising administering a vascular endothelial growth factor (VEGF) antagonist or a VEGF-receptor (VEGFR) antagonist to the same eye after wash out of the monospecific placental growth factor (PlGF) antagonist previously administered to the eye.

8. The method of claim 1, further comprising administering the monospecific placental growth factor (PlGF) antagonist to an eye in combination with a second active compound, wherein said second active compound is different from a vascular endothelial growth factor (VEGF) antagonist and different from a VEGF-receptor (VEGFR) antagonist.

9. The method of claim 1, further comprising administering the monospecific placental growth factor (PlGF) antagonist to an eye in combination with a second active compound, wherein said second active compound is different from a vascular endothelial growth factor (VEGF) antagonist and different from a VEGF-receptor (VEGFR) antagonist; and wherein said administration is after wash out of a vascular endothelial growth factor (VEGF) antagonist or VEGF-receptor (VEGFR) antagonist previously administered to the same eye.

10. The method of claim 1, wherein the monospecific placental growth factor (PlGF) is administered to an eye in combination with a second active compound wherein said second active compound is different from a vascular endothelial growth factor (VEGF) antagonist and different from a VEGF-receptor (VEGFR) antagonist, and further comprising administering a vascular endothelial growth factor (VEGF) antagonist or a VEGF-receptor (VEGFR) antagonist to the same eye after wash out of the monospecific placental growth factor (PlGF) antagonist previously administered to the eye in combination with the second active compound.

11. The method of claim 8, wherein the monospecific placental growth factor (PlGF) antagonist and said second active compound are administered to the eye each in a separate composition.

12. The method of claim 8, wherein the monospecific placental growth factor (PlGF) antagonist and said second active compound are administered to the eye combined in a single composition.

13. The method of claim 8, wherein said second active compound is an anti-inflammatory compound, an anti-angiogenic compound, an anti-fibrotic compound, an AGE-inhibiting compound, an ALE-inhibiting compound, an AGE-breaking compound, a carbonic anhydrase inhibitor, an NMDA-receptor antagonist, a kainate receptor antagonist, an AMPA-receptor antagonist, a neuroprotective agent, an agent for controlling the intra-ocular pressure, an anti-apoptotic agent, an antiviral compound, an antibiotic compound, an antifungal compound, an antihistamine, an anticoagulant, a thrombolytic compound, an anti-mitotic agent, an anesthetic agent, and agent inducing mydriasis, an agent inducing cycloplegia, an agent inducing posterior vitreous detachment (complete or incomplete), an agent inducing vitreous liquefaction, an integrin inhibitor, an anti-edema agent.

14. The method of claim 1, further comprising photodynamic therapy, laser photocoagulation, radiation therapy or vitreal surgery.

15. The method of claim 1, wherein the posterior ocular fibrosis is occurring concurrent with or after retinal damage.

16. The method of claim 1, wherein the posterior ocular fibrosis is occurring in age-related macular edema, diabetic retinopathy, (diabetic) macular edema, any type of retinopathy, neovascular glaucoma, retinal detachment or retinal hemorrhage.

17. The method of claim 1, wherein the monospecific placental growth factor (PlGF) antagonist is a PlGF-neutralizing antibody or a PlGF-neutralizing fragment of an antibody, an antisense RNA, a small interfering RNA, an aptamer, or a ribozyme.

18. The method of claim 1, wherein the monospecific placental growth factor (PlGF) antagonist is a PlGF-neutralizing antibody or a PlGF-neutralizing antibody fragment comprising the 6 complementarity determining regions (CDRs) as defined in SEQ ID NO: 1 to 6.

19. The method of claim 1, wherein the monospecific placental growth factor antagonist is an isolated PlGF-neutralizing antibody or a PlGF-neutralizing antibody fragment thereof comprising the 6 CDRs as defined in SEQ ID NO: 14 to 19.

20. The method of claim 1, wherein the subject has age-related macular degeneration and a posterior ocular fibrotic scar developed after anti-VEGF treatment.
